SEQUENCE LISTING (25 sequences)

SEQ ID NO: 1 - DNA, 24 bp, Artificial Sequence
  Description of Artificial Sequence Synthetic primer
gcagagctctctggctaactagag

SEQ ID NO: 2 - DNA, 80 bp, Artificial Sequence
  Description of Artificial Sequence Synthetic primer
ttttttttttttttttttttttttttttttttttttttttttttttttttttttttttttctggcaactagaaggcacag

SEQ ID NO: 3 - DNA, 44 bp, Artificial Sequence
  Description of Artificial Sequence Synthetic primer
  modified_base (21)..(24): a, c, t, g, unknown or other
gacgtgtgctcttccgatctnnnnccggttaatgtggctctggt

SEQ ID NO: 4 - DNA, 44 bp, Artificial Sequence
  Description of Artificial Sequence Synthetic primer
  modified_base (21)..(24): a, c, t, g, unknown or other
acacgacgctcttccgatctnnnngactaggaaggaggaggcct

SEQ ID NO: 5 - DNA, 128 bp, Artificial Sequence
  Description of Artificial Sequence Synthetic polynucleotide
  modified_base (1)..(4): a, c, t, g, unknown or other
  modified_base (125)..(128): a, c, t, g, unknown or other
nnnngactaggaaggaggaggcctaaggatggggcttttctgtcaccaatcctgtccctagtggccccactgtggggtggaggggacagataaaagtacccagaaccagagccacattaaccggnnnn

SEQ ID NO: 6 - DNA, 24 bp, Artificial Sequence
  Description of Artificial Sequence Synthetic primer
ctgtgcttcaaggtccttgtctgc

SEQ ID NO: 7 - DNA, 24 bp, Artificial Sequence
  Description of Artificial Sequence Synthetic primer
ctctgtctccttctacagccaagc

SEQ ID NO: 8 - DNA, 23 bp, Artificial Sequence
  Description of Artificial Sequence Synthetic primer
ctgcctcataaggttgccctaag

SEQ ID NO: 9 - DNA, 29 bp, Artificial Sequence
  Description of Artificial Sequence Synthetic primer
ccagcaatagatgatccaactcaaattcc

SEQ ID NO: 10 - DNA, 46 bp, Artificial Sequence
  Description of Artificial Sequence Synthetic primer
  modified_base (21)..(25): a, c, t, g, unknown or other
acacgacgctcttccgatctnnnnngccaggttgagcaggtagatg

SEQ ID NO: 11 - DNA, 46 bp, Artificial Sequence
  Description of Artificial Sequence Synthetic primer
agacgtgtgctcttccgatctgctctactcactggtgttcatcttt

SEQ ID NO: 12 - DNA, 25 bp, Artificial Sequence
  Description of Artificial Sequence Synthetic primer
gctctactcactggtgttcatcttt

SEQ ID NO: 13 - DNA, 48 bp, Artificial Sequence
  Description of Artificial Sequence Synthetic primer
  modified_base (21)..(24): a, c, t, g, unknown or other
acacgacgctcttccgatctnnnncctcttggttttacagatacgaac

SEQ ID NO: 14 - DNA, 39 bp, Artificial Sequence
  Description of Artificial Sequence Synthetic primer
gacgtgtgctcttccgatctctcacctcagctggaccac

SEQ ID NO: 15 - DNA, 133 bp, Artificial Sequence
  Description of Artificial Sequence Synthetic polynucleotide
  modified_base (1)..(4): a, c, t, g, unknown or other
nnnncctcttggttttacagatacgaacctaaactttcaaaacctgtcagtgattgggttccgaatcctcctcctgaaagtggccgggtttaatctgctcatgacgctgcggctgtggtccagctgaggtgag

SEQ ID NO: 16 - DNA, 20 bp, Artificial Sequence
  Description of Artificial Sequence Synthetic primer
cggaactctgccctctaacg

SEQ ID NO: 17 - DNA, 24 bp, Artificial Sequence
  Description of Artificial Sequence Synthetic primer
gtgtgtcaccagataaggaatctg

SEQ ID NO: 18 - DNA, 19 bp, Artificial Sequence
  Description of Artificial Sequence Synthetic primer
cgtctctctcctgagtccg

SEQ ID NO: 19 - DNA, 59 bp, Artificial Sequence
  Description of Artificial Sequence Synthetic primer
  modified_base (31)..(34): a, c, t, g, unknown or other
ctctttccctacacgacgctcttccgatctnnnnctctggttctgggtacttttatctg

SEQ ID NO: 20 - DNA, 45 bp, Artificial Sequence
  Description of Artificial Sequence Synthetic primer
agacgtgtgctcttccgatctgtgtgtcaccagataaggaatctg

SEQ ID NO: 21 - DNA, 171 bp, Artificial Sequence
  Description of Artificial Sequence Synthetic polynucleotide
  modified_base (1)..(4): a, c, t, g, unknown or other
nnnnctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacac

SEQ ID NO: 22 - DNA, 182 bp, Artificial Sequence
  Description of Artificial Sequence Synthetic polynucleotide
  modified_base (1)..(4): a, c, t, g, unknown or other
nnnnctctggttctgggtacttttatctgtcccctccaccccacagtggggcaagcttcgagccatcagggcctggttctttccgcctcagaaggccttttgcagtttatcaggatgaggatgaccagcatgttgcccacaaaaccaaagatgaacaccagattccttatctggtgacacac

SEQ ID NO: 23 - DNA, 59 bp, Artificial Sequence
  Description of Artificial Sequence Synthetic primer
  modified_base (34)..(38): a, c, t, g, unknown or other
acactctttccctacacgacgctcttccgatctnnnnngccaggttgagcaggtagatg

SEQ ID NO: 24 - DNA, 122 bp, Artificial Sequence
  Description of Artificial Sequence Synthetic polynucleotide
  modified_base (1)..(5): a, c, t, g, unknown or other
nnnnngccaggttgagcaggtagatgtcagtcatgctcttcagccttttgcagtttctcgagccatcagggcctggttctttccgcctcagaagtagaaagatgaacaccagtgagtagagc

SEQ ID NO: 25 - DNA, 119 bp, Artificial Sequence
  Description of Artificial Sequence Synthetic polynucleotide
  modified_base (1)..(5): a, c, t, g, unknown or other
nnnnngccaggttgagcaggtagatgtcagtcatgctcttcagccttttgcagtttatcaggatgaggatgaccagcatgttgcccacaaaaccaaagatgaacaccagtgagtagagc

(19) United States
(12) Patent Application Publication
(10) Pub. No.: US 2019/0352614 A1
(43) Pub. Date: Nov. 21, 2019

(54) B-CELL ENGINEERING

(71) Applicant: Sangamo Therapeutics, Inc., Richmond, CA (US)

(72) Inventors: Rainier Amora, Richmond, CA (US); Michael C. Holmes, Richmond, CA (US); Brigit E. Riley, Richmond, CA (US)

(21) Appl. No.: 16/480,939

(22) PCT Filed: Jan. 25, 2018
(86) PCT No.: PCT/US2018/015180

§ 371 Date: Jul. 25, 2019

Related U.S. Application Data
(60) Provisional application No. 62/450,917, filed on Jan. 26, 2017.

(51) Int. Cl. C12N 5/16; C12N 15/90; A61K 35/17; A61K 35/28; A61K 38/48

(52) U.S. Cl. CPC C12N 5/163; C12N 15/907; A61K 38/4846; A61K 35/28; A61K 35/17

(57) ABSTRACT

Described herein are constructs used for B-cell genomic engineering and for expression of a transgene and/or for modulation of B cell function.

### CROSS-REFERENCE TO RELATED APPLICATIONS

The present application claims the benefit of U.S. Provisional Application No. 62/450,917 filed Jan. 26, 2017, the disclosure of which is hereby incorporated by reference in its entirety.

### TECHNICAL FIELD

The present disclosure is in the field of gene therapy, particularly genome editing and targeted delivery of transgene-encoding constructs to B cells for expression of beneficial (therapeutic) proteins.

### BACKGROUND

Gene therapy can be used to genetically engineer a cell to have one or more inactivated genes and/or to cause that cell to express a product not previously being produced in that cell (e.g., via transgene insertion and/or via correction of an endogenous sequence). Examples of uses of transgene insertion include nuclease-mediated modification including the insertion of one or more genes encoding one or more novel therapeutic proteins, including therapeutic antibodies, insertion of a coding sequence encoding a protein that is lacking in the cell or in the individual, insertion of a wild type gene in a cell containing a mutated gene sequence, and/or insertion of a sequence that encodes a structural nucleic acid such as a microRNA or siRNA. These techniques can also be used to knock out expression of an endogenous gene and/or to alter the toxicity profile of the cell. See, e.g., U.S. Pat. Nos. 9,394,545; 9,150,847; 9,206,404; 9,045,763; 9,005,973; 8,956,828; 8,936,936; 8,945,868; 8,871,905; 8,586,526; 8,563,314; 8,329,986; 8,399,218; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 20100218264; 20120017290; 20110265198; 20130137104; 20130122591; 20130177983 and 20130177960 and 20150056705.

B cells function in humoral immunity by secreting antibodies against a variety of antigens. They are professional antigen presenting cells (APC) and are activated by helper T cells to differentiate into plasma cells that produce large amounts of antigen-specific antibodies. B cell development takes place in both the fetal liver and the bone marrow, and a critical step in B cell development is the generation of a B cell receptor (BCR), a complex structure comprising unique heavy and light chains. The process of BCR generation includes the rearrangement of the various immunoglobulin (Ig) gene segments in both the heavy and light chains of the BCR genes during the pro-B cell phase of B cell maturation. Pro-B cells become pre-B cells following successful pairing of rearranged heavy and light chains where the pre-BCR produced is expressed on the cell surface of the pre-B cells. Signaling through the pre-BCR drives further B cell development leading the pre-B cells to enlarge. Eventually, the large pre-B cells stop proliferating and additional light chain rearrangement occurs leading to the expression of a unique IgM BCR on the cell surface of what is now considered an immature B cell. These cells then exit the bone marrow and circulate in the periphery as transitional B cells.

Maturation of the immature B cells occurs primarily in the spleen where selection also occurs such that B cells producing antibodies with high affinity to self-antigens will be destroyed (see Naradikian et al (2014) in Drugs Targeting B-Cells in Autoimmune Diseases, Milestones in Drug Therapy (X. Bosch et al (eds)) doi 10.1007/978-3-3-0348-0706-7_2, Springer Basel). Circulating B cells are able to enter secondary lymph nodes and spleen and acquire antigen from follicular dendritic cells. Upon entry into the lymph node/spleen and interaction with the dendritic cells, the B cells internalize the antigen and it is processed such that peptide fragments of the antigen are presented via MHC class II molecules to the cognate CD4+ T cells. These T cells have been previously activated by an APC presenting the same antigen. The interaction between B and T cells leads to a number of events, including the full activation of the T cell, resulting in T cell proliferation. T cells then produce cytokines that act directly on the B cells to induce B cell proliferation and class switching of the antibody expressed on the B cell surface. The proliferating B cells cluster in transient regions within the lymph nodes and spleen known as ‘Germinal Centers’. These activated B cells differentiate into either a specialized antibody secreting cells (plasmablast or plasma cell) that appear to undergo a pre-programmed number of divisions (typically 5-6) before they complete their final stage of differentiation to become non-proliferating plasma cells. Alternatively, the activated B cells leave the Germinal Center and differentiate into memory B cells (Zhang et al, (2016) Immunol Rev 270(1): 8-19).

Following activation, the genome mutator enzyme activation-induced cytidine deaminase (AID) is expressed which leads to somatic hypermutation in the antibody genes and class-switch recombination (CSR) altering antibody effector function. The somatic hypermutation occurs in the immunoglobulin variable region (IgV) gene to generate a repertoire of antibody mutants with varying affinities to the antigen (Klein and Heise (2015) Curr Opin Hematol 22(4): 379-387). This process takes place in the so-called ‘dark zone’ within the Germinal Center. The differentiated B cells migrate into the ‘light zone’ where B cells producing higher-affinity antibodies compete for available antigen and/or T cell help such that they receive survival signals through their B cell receptors. Lower affinity antibody producing B cells do not receive these survival signals because they cannot compete with their higher-affinity producing B cell siblings, and so they undergo apoptosis. The higher affinity antibody producing B cells can then either re-enter the dark zone for additional rounds of proliferation and somatic hypermutation, can leave the Germinal Center and differentiate into plasmablasts or can differentiate into long-lived memory B cells (Recaldin and Fear (2015) Clin and Exp Immunol 183:65-75).

Thus, in a very complex process, B cells are induced to express large amounts of antibodies against antigens for protection of the body from a number of potential threats. Interestingly, there are a number of pathogens (e.g. parasites, bacteria, viruses) that are able to subvert the antibody response. These pathogens include a number of agents responsible for a great deal of human disease including but not limited to Plasmodium, Schistosomia, Mycobacterium, HIV, HCV and HBV (Borhis and Richard (2015) BMC Immunology 16:15 doi 10.1186/s12865-015-0079-y). The mechanisms at play behind pathogen-mediated suppression of the antibody response are not all known, but it appears that certain pathogens induce production of unusual B cell subtypes, which B cell subtypes alter the cellular microenvironment, leading to suppression of both B and T cells. For example, HBV has been shown to interfere with stimulation through the Toll-like receptor 9 (TLR9) such that dendritic cells produce reduced IFN-α (known to induce B cells to proliferate and secrete IgMs). It appears that HBV can selectively inhibit TLR9 expression in B lymphocytes (Vincent et al (2011) PLoS ONE 6(10):e26315. doi: 10.1371/journal.pone.0026315).

Over the past decade, studies have provided well-founded evidence in both mice and humans of discrete subsets of immunoregulatory B cells. These suppressor B cells have the capacity to maintain immune tolerance and to suppress pathological autoimmune and inflammatory immune responses, as well as to suppress responses during cancer immune surveillance, through the release of anti-inflammatory mediators, such as interleukin-10 (IL-10) and the expression of inhibitory molecules, such as PD-L1. These studies have led to the conclusion that there is a pool of B cells that have a suppressor role in immune tolerance, and this pool is now referred to as regulatory B cells or “Bregs”. Other phenotypes associated with human Bregs include suppression of autoimmune inflammation and a role in allergen tolerance. More recent studies have demonstrated that B cells can play contradictory roles in cancer progression. For example, IL-10-producing CD1d^{high}CD5+ B cells isolated from CLL patients treated with rituximab revealed that anti-CD20-mediated B-cell depletion mostly enriched a Breg pool. The enriched Bregs were postulated to suppress the anti-tumor immunity required for the clearance of anti-CD20-bound tumor cells, causing patients to develop lymphoma resistance towards anti-CD20 therapy and/or eventually relapse as a result of enhanced cancer progression (Bodogai et al, (2013) Cancer Res 73:2127-2138).

Findings that human B cells negatively modulate tumor growth were noted when the presence of CD20+ B-cell tumor-infiltrating lymphocytes in ovarian cancer, non-small lung carcinoma and cervical cancer correlated with improved survival and lower relapse rates. These studies showed that tumor-infiltrating B cells correlated with favorable outcomes. Bregs can suppress diverse cell subtypes, including T cells, through the secretion of anti-inflammatory mediators, such as IL-10, and can facilitate the conversion of T cells to regulatory T cells, thus attenuating anti-tumor immune responses. The potential mechanisms underlying B-cell anti-tumor immunity may involve the secretion of effector cytokines, such as IFN-γ, by B cells, which could polarize T cells towards a Th1 or Th2 response or promote T-cell responses through their role as antigen-presenting cells (Sarvaria et al (2017) Cell Mol Immunol 14(8):662-674).

However, human B cells have also been shown to foster tumor progression. The presence B cells with activated STAT3 in human tumor tissues was could to correlate with the severity of tumor angiogenesis. Also, infiltration of CD19+ B cells in patients with metastatic ovarian carcinoma; or the increased infiltration of CD20+ and CD138+ B cells in patients with epithelial ovarian cancer is associated with poor disease prognosis and outcome. Further, reduced tumor burden following partial B cell reduction with rituximab was found in 50% of patients with advanced colorectal cancer. Thus, the exact role that Bregs play in cancer is still unclear, but most likely relates to the activity of subtypes of Bregs (Sarvaria, ibid).

A considerable number of disorders are either caused by an insufficiency of a secreted gene product or are treatable by secretion of a therapeutic protein. Clotting disorders, for example, are fairly common genetic disorders where factors in the clotting cascade are aberrant in some manner, i.e., lack of expression or production of a mutant protein. Most clotting disorders result in hemophilias such as hemophilia A (factor VIII deficiency), hemophilia B (factor IX deficiency), or hemophilia C (factor XI deficiency). Treatment for these disorders is often related to the severity. For mild hemophilias, treatments can involve therapeutics designed to increase expression of the under-expressed factor, while for more severe hemophilias, therapy involves regular infusion of the missing clotting factor (often 2-3 times a week via enzyme replacement therapy (ERT)) to prevent bleeding episodes. Patients with severe hemophilia are often discouraged from participating in many types of sports and must take extra precautions to avoid everyday injuries.

Alpha-1 antitrypsin (A1AT) deficiency is an autosomal recessive disease caused by defective production of alpha 1-antitrypsin which leads to inadequate A1AT levels in the blood and lungs. It can be associated with the development of chronic obstructive pulmonary disease (COPD) and liver disorders. Currently, treatment of the diseases associated with this deficiency can involve infusion of exogenous A1AT and lung or liver transplant.

Lysosomal storage diseases (LSDs) are a group of rare metabolic monogenic diseases characterized by the lack of functional individual lysosomal proteins normally involved in the breakdown of waste lipids, glycoproteins and mucopolysaccharides. These diseases are characterized by a buildup of these compounds in the cell since it is unable to process them for recycling due to the mis-functioning of a specific enzyme. Common examples include Gaucher's (glucocerebrosidase deficiency—gene name: GBA), Fabry's (α galactosidase deficiency—GLA), Hunter's (iduronate-2-sulfatase deficiency-IDS), Hurler's (alpha-L iduronidase deficiency—IDUA), and Niemann-Pick's (sphingomyelin phosphodiesterase Ideficiency—SMPD1) diseases.

Type I diabetes is a disorder in which immune-mediated destruction of pancreatic beta cells results in a profound deficiency of insulin, which is the primary secreted product of these cells. Restoration of baseline insulin levels provide substantial relief from many of the more serious complications of this disorder which can include “macrovascular” complications involving the large vessels: ischemic heart disease (angina and myocardial infarction), stroke and peripheral vascular disease, as well as “microvascular” complications from damage to the small blood vessels. Microvascular complications may include diabetic retinopathy, which affects blood vessel formation in the retina of the eye, and can lead to visual symptoms, reduced vision, and potentially blindness, and diabetic nephropathy, which may involve scarring changes in the kidney tissue, loss of small or progressively larger amounts of protein in the urine, and eventually chronic kidney disease requiring dialysis.

However, provision of therapeutic proteins to treat disorders in a subject may be limited by the subject's own immune response to the therapeutic protein, including the production of antibodies by B-cells in the subject, which may limit the efficacy of such treatments. For example, hemophilia patients receiving ERT of the clotting factor(s) in which they are deficient or lacking (e.g., Factor VIII, Factor IX, etc.) may develop antibodies to these needed proteins (e.g., anti-F9 antibodies). It is estimated that 15-50% of hemophilia A patients develop inhibitory antibodies against therapeutic Factor 8 protein (Krudysz-Amblo et al, (2009) Blood 113(11):2587-2594). In some cases, the reactions may be severe (anaphylactic shock) leading to a situation where the needed ERT causes harmful side-effects in the patient (see e.g. J M Lusher (2000) Semin Thromb Hemost 26(2): 179-188).

In addition, antibodies are secreted protein products whose binding plasticity has been exploited for development of a diverse range of therapies. Therapeutic antibodies can be used for neutralization of target proteins that directly cause disease (e.g. VEGF in macular degeneration) as well as for highly selective killing of cells whose persistence and replication endanger the host (e.g. cancer cells, as well as certain immune cells in autoimmune diseases, including B cells that produce that antibodies to self-antigens). In such applications, therapeutic antibodies take advantage of the body's normal response to its own antibodies to achieve selective killing, neutralization, or clearance of target proteins or cells bearing the antibody's target antigen. Thus, antibody therapy has been widely applied to many human conditions including oncology, rheumatology, transplant, and ocular disease.

Thus, there remains a need for additional methods and compositions that can be used to express a desired transgene at a therapeutically relevant level in a subject to treat genetic diseases such as hemophilias, diabetes, lysosomal storage diseases and/or A1AT deficiency, including treating and/or avoiding any associated toxicity and which may limit expression of the transgene or therapeutic protein to the desired tissue type, including by limiting innate B cell responses. Additionally, there remains a need for additional methods and compositions to express a desired transgene (for example an antibody) at a therapeutically relevant level for the treatment of other diseases such as cancers.

### SUMMARY

Site-specific modification of B cells at one or more genetic loci would enhance B-cell function (including enhancing antibody production by these cells and/or targeting B-cells to produce proteins that limit unwanted innate immune responses), differentiation into plasmablasts and engraftment capabilities. Controlling B-cells via target genetic modification (e.g., disruption and/or genomic or epiosomal gene addition) allows for efficient and less toxic protein replacement therapies and in addition allows communication within Germinal Centers to be programmed.

The present invention describes compositions and methods for modulating expression of a target gene in a B cell and/or expressing a transgene in a B cell (including derivative plasmablast or plasma cells). Thus, provided herein are genetically modified B cells (including B cells descended from genetically modified hematopoietic stem cells or other B cell prescursors) comprising one or more of the following modifications: inclusion of one or more transgenes in the cell; and/or insertions and/or deletions which modify (i) B cell receptor genes, and/or (ii) cellular interactions in Germinal Centers; and/or (c) modifications that inhibit suppression of any B cell function associated with pathogen infection or cancer regulation. The transgene(s) may be expressed extra-chromosomally (episomally) and/or may be integrated into the genome of the B cell (e.g., via nuclease-mediated targeted integration, for example into a safe harbor locus). In some embodiments, one or more transgenes are maintained episomally and one or more transgenes are integrated into the genome of the cell (B cell or HSC that is differentiated into a B cell). In some embodiments, the transgene encodes a protein involved in the clotting cascade. In other embodiments, the transgene encodes an enzyme defective in a lysosomal storage disorder or encodes a therapeutic antibody. In other embodiments, the transgene encodes a molecule that targets a B cell producing an undesirable antibody, for example, an antibody against a therapeutic protein, including but not limited to antibodies against an endogenous protein (e.g., autoantibodies in an autoimmune diseases) and/or an exogenous protein (e.g., a protein supplied by ERT such as a clotting factor). For example, the transgene can encode an antibody that recognizes the B cell receptor on B cells that are sensitive to the desirable protein (endogenous protein in autoimmune disease and/or ERT-supplied protein) to target the B cell population that is producing the undesirable antibodies against the desirable protein. Non-limiting examples of such antibodies include antibodies that recognize a B cell receptor associated with a B cell producing antibodies against ERT-supplied proteins such as clotting factors in hemophilia (e.g., B-cells that product anti-F9 or anti-F8 antibodies); and/or recognize a B cell receptor on a B cell producing antibodies against auto/self-antigens including but not limited to myelin basic protein (MBP) in MS, Antinuclear antibodies (ANAs) in systemic lupus erythematosus (SLE), glycoproteins in the heart, joint and other tissues in acute rheumatic fever, antibodies to Fc portion of IgG in rheumatoid arthritis (RA), as well as B cells producing autoantibodies in Reiter's syndrome, Sjogren's syndrome, Systemic sclerosis (Scleroderma), Inflammatory myopathies, Polyarteritis nodos, Graves Disease, Type I diabetes and the like. The compositions and methods described herein result in high levels of protein production both in vitro and in vivo, including at levels sufficient to show clinically relevant (therapeutic) effects in vivo.

In one aspect, described herein is a polynucleotide expression construct comprising at least one B cell specific promoter, which promoter drives expression of one or more transgenes. The B cell promoter can be selected from any promoter that is active in B cells, including but not limited to the immunoglobulin kappa chain promoter (Igκ, Laurie et al (2007) Gene Ther 14(23): 1623-31), B29 (Hermanson et al (1989) Proc Nat'l Acad Sci 86: 7341-7345), BCL6 (Ramachandrareddy et al (2010) Proc Nat'l Acad Sci 107(26): 11930-11935), CIITA promoter III (Deffernnes et al (2001) J. Immunol 167(1): 98-106), mb-1 (see e.g. Malone and Wall (2001) J. Immunol 168(7):3369-3375) and the EEK promoter, comprising the light chain promoter (VKp) preceded by an intronic enhancer (iEκ), an MAR, and a 3′ enhancer (3′ Eκ) (see U.S. Pat. No. 8,133,727). In some embodiments, the B cell specific promoter used is normally expressed in the Germinal Center such that the transgene is expressed when the cell is in a Germinal Center (e.g. BCL6, Basso et al (2010) Blood 115(5):975-984). In some embodiments, the transgene is inserted via nuclease-mediated targeted integration such that it is controlled by the B cell specific promoter in the genome of the cell. In other embodiments, the B cell promoter-transgene construct is part of a DNA vector that is maintained extra-chromosomally. In some embodiments, the B cell promoter-transgene construct is inserted in a transcriptionally silent and/or safe harbor region of the B cell genome such as into an albumin gene or a gene encoding a subunit of the T cell receptor (e.g., TCRA or TCRB).

In some aspects, the transgene encodes an enzyme that is lacking or insufficient in subject. In some embodiments, the transgene encodes a clotting factor such as Factor VII, Factor VIII, Factor IX, Factor X, Factor XI or Factor XII. In other embodiments, the transgene encodes an enzyme deficient in a lysosomal storage disease, including but not limited to glucocerebrosidase (GBA), a galactosidase (GLA), β-glucuronidase (GUSB), iduronate-2-sulfatase (IDS), alpha-L iduronidase (IDUA), sphingomyelin phosphodiesterase 1 (SMPD1), or alpha-glucosidase (GAA). In some embodiments, the transgene encodes A1AT. Non-limiting examples of proteins that may be expressed as described herein also include fibrinogen, prothrombin, tissue factor, Factor V, von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (ornithine transcarbamylase), ASS (argininosuccinic acid synthetase), ASL (argininosuccinase acid lyase) and/or ARG1 (arginase), and/or a solute carrier family 25 (SLC25A13, an aspartate/glutamate carrier) protein, a UGT1A1 or UDP glucuronsyltransferase polypeptide A1, a fumarylacetoacetate hydrolyase (FAH), an alanine-glyoxylate aminotransferase (AGXT) protein, a glyoxylate reductase/hydroxypyruvate reductase (GRHPR) protein, a transthyretin gene (TTR) protein, an ATP7B protein, a phenylalanine hydroxylase (PAH) protein, a lipoprotein lyase (LPL) protein, an engineered nuclease, an engineered transcription factor and/or an engineered single chain variable fragment antibody (diabody, camelid, etc.). In one preferred embodiment, the transgene encodes a FVIII polypeptide. In some embodiments, the FVIII polypeptide comprises a deletion of the B domain. In some embodiments, provided herein are methods and compositions to express therapeutically relevant levels of one or more therapeutic proteins from one or more transgenes. In certain embodiments, expression of a transgene construct encoding a replacement protein results in 1% of normal levels of the protein produced, while in others, 2%, 3%, 4%, 5%, 10%, 15%, 20%, 30%, 50%, 80%, 100%, 150%, 200%, or more of normal levels of the protein are produced. In some embodiments, the transgene encodes a polypeptide that circumvents the inhibition of the antibody response by virus, bacteria or parasite.

CD19-positive B cells differentiated in vitro to plasmablasts and plasma cells produce upwards of 10,000 ng/mL of antibodies (IgG, IgM, IgA). Thus, in some aspects, the transgene encodes a therapeutic protein such as a single chain antibody. In some embodiments, the single chain antibody is a scFv while in others, the single chain antibody is a camelid antibody or nanobody (see e.g. Mejias et al (2016) Sci Reports 6: srep24913, doi: 10:1038). In other aspects, more than one transgene is expressed in the B cell. In one embodiment, the more than one transgenes include the sequences necessary to express a full antibody or fragment thereof, or another antigen-binding protein (e.g. monobody, aptamer, darpin, adnectins, affibodies, anticalins, kunitz-type inhibitors etc. (Gebauer and Skerra (2009) Curr Opin Chem Biol 13(3):245-55).

In some embodiments, a population of B cells comprising a transgene encoding a therapeutic protein of interest is engineered ex vivo and then re-introduced into a subject in need thereof. The B cell population may be engineered as described herein at any stage of development, including but not limited to as a hematopoietic stem cell (HSC), a lymphoid progenitor cell or a mature B cell. Stem or progenitor of B cells may be engineered and then differentiated in vitro and administered as progenitor (lineage-committed B cells) or mature cells to a subject. Alternatively, engineered stem or B cell progenitor cells may be engineered in vitro as described herein and fully differentiated into mature B cells in vivo following administration. Thus, for ex vivo administrations, the B cell populations as described herein may be heterogenous in that they include stem, progenitor and/or mature B cells are various stages of development. Alternatively, the populations of B cells may homogenous and include only stem, progenitor or mature cells. In still other embodiments, the engineered B cells are made in vivo using a delivery vector that can transduce B cells. In further embodiments, the delivery vector is a viral vector, preferably an adeno associate virus (AAV). In preferred embodiments, the AAV vector is an AAV6 vector. In other embodiments, the delivery vector is non-viral, for example mRNA, a lipid nanoparticle (LNP) or plasmid vector.

In a further aspect, engineered B cells as described herein (including populations of B cells) are grown in vitro for the production of a protein encoded by a transgene. In preferred embodiments, the protein is an antibody or antigen binding protein (e.g., an antibody that binds to endogenous B cells producing undesirable antibodies in the subject, including endogenous B cells producing antibodies against ERT-supplied proteins such as clotting factors and/or B cells producing antibodies against self-proteins in autoimmune disorders), or an antibody that neutralizes the unwanted antibodies. The protein produced from the B cells may be isolated and used for protein therapy such as enzyme replacement therapy and/or in conjunction with enzyme replacement therapy to reduce and/or eliminate innate production of undesirable antibodies (e.g., anti-ERT antibodies developed following ERT).

In some aspects, the invention provides methods and compositions to deliver a B cell or plasma cell that expresses a transgene that crosses the blood brain barrier, useful for the treatment and/or prevention of a disease of or which impacts the CNS. In some embodiments, the transgene encodes an enzyme lacking in a subject with a lysosomal storage disorder. In further embodiments, the transgene encodes glucocerebrosidase (GBA), a galactosidase (GLA), β-glucuronidase (GUSB), iduronate-2-sulfatase (IDS), alpha-L iduronidase (IDUA), sphingomyelin phosphodiesterase 1 (SMPD1), or alpha-glucosidase (GAA) and is used to treat or prevent the CNS disease associated with Gaucher disease (Bae et al, (2015) Exp Mol Med 47, e153; doi:10:1038/emm.2014.128), Fabry disease, MPS type VII (Sly et al (1973) J Pediatr. 1973 February; 82(2):249-57), MPS II, MPS I, Niemann-Pick or Pompe disease, respectively.

In further aspects, the methods and compositions of the invention include a modified B cell or B cell derivative (plasmablast, plasma cell) comprising a transgene and also one or more further modifications. The further modification may be additional episomal or additional integrated sequences (which may be integrated at the same and/or at different locations in the genome). In some embodiments, the transgene-comprising B cells further comprise additional protein or peptide sequences (or polynucleotides encoding the same) that aid in the efficiency of crossing of the blood brain barrier. In some embodiments, the peptide comprises a peptide known in the art to facilitate crossing into the brain. In further embodiments, the peptide is an antibody expressed on the B cell surface that targets the transferrin receptor, while in other embodiments, the peptide is a metallotransferrin (Karkan et al (2008) PLoS ONE 3(6):e2469. doi:10.1371/journal.poine.0002469. In some embodiments, the peptide is a receptor such as VLA-4, ICAM-1, IL-8Ra (CXCR1), or IL-8Rb (CXCR2) (Alter et al (2003) J. Immunol 170:4497-4505). In any of these embodiments, the transgene may encode an enzyme lacking in a lysosomal storage disease such as those described above such that the enzyme is delivered into the CNS of a subject in need thereof.

In some aspects, the engineered B cells of the invention comprise further modifications (e.g., mutations) that aid in engraftment after transplant. In some embodiments, expression of specific genes is inhibited (e.g., via transient repression or permanent knock-out) to increase engraftment and/or size of the germinal center. Genes subject to such inhibition include, but are not limited to, inositol hexakisphosphate kinases (Zhang et al (2014) Basic Res Cardio 109(4): 417), Glycogen synthase kinase-3β (GSK-3β, see Ko et al (2011) Stem Cells 29(1):108-18), CD26 (DPPIV/dipeptidylpeptidase IV) peptidase, (Tian et al (2006) Gene Ther 13(7):652-8), RhoA (Ghiaur et al (2006) Blood 108(6):2087-94), EAF2 (Li et al., (2016) Nat Com 7; doi: 10.1038/ncomms10836), autophagy proteins such as Atg5 (Pengo et al., (2013) Nat Immunol 14(3):298-305) and the like. In further embodiments, the engineered B cells may be further engineered to repress (e.g., knock out) genes associated with induction of a graft-versus-host reaction. In some embodiment, genes encoding the B cell receptor are knocked out to prevent stimulation of a B cell in a host.

In other aspects, the engineered B cells of the invention comprise further modification (mutations such as genomic insertions and/or deletions; episomal expression of transgenes, etc.) which regulate cellular interactions (e.g., T cell-B cell interactions) in Germinal Centers and/or inhibit suppression of any B cell function (e.g., antibody production, cytokine expression, signaling, etc.) associated with pathogen infection. In some aspects, the engineered B cells of the invention further comprise proteins (or sequences encoding these proteins) for the inhibition of B cells that are involved in oncogenic behavior. For example, in some embodiments, the engineered B cells comprise a surface expressed antibody against ubiquitin hydrolase UCH-L1 (including a transgene encoding the same) to suppress B cells involved in some types of large B-cell lymphoma (Bedekovics et al (2016) Blood 127(12):1564-74).

In another aspect, pharmaceutical compositions comprising one or more of the cells, expression constructs and/or optional nucleases described herein are provided.

For nuclease-mediated targeted integration of the expression constructs of the present invention into a suitable location in a B cell, any nuclease can be used, including but not limited to, one or more zinc finger nucleases (ZFNs), TALENs, CRISPR/Cas nucleases and/or TtAgo nucleases, such that the expression construct is integrated into the region (gene) cleaved by the nuclease(s). In certain embodiments, one or more pairs of nucleases are employed. The nucleases may be introduced in mRNA form or may be administered to the cell using non-viral or viral vectors. In some aspects, the nuclease polynucleotides may be delivered by lentivirus or by non-integrating lentivirus. In other aspects, the expression cassette may be delivered by AAV and/or DNA oligos.

In any of the compositions and methods described, expression cassettes and/or nucleases may be carried on an AAV vector, including but not limited to AAV1, AAV3, AAV4, AAV5, AAV6, AAV8, AAV9 and AAVrh10 or pseudotyped AAV such as AAV2/8, AAV8.2, AAV2/5 and AAV2/6 and the like. In certain embodiments, the polynucleotides (expression constructs and/or nucleases) are delivered using the same AAV vector types. In other embodiments, the polynucleotides are delivered using different AAV vector types. The polynucleotides may be delivered using one or more vectors. In further embodiments, the polynucleotides are delivered via a lipid nanoparticle (LNP). In certain embodiments, the polynucleotides are delivered via administration into the spleen or lymph node of an intact animal. In other embodiments, the polynucleotides are delivered via intravenous administration in a peripheral vein.

The methods described herein can be practiced in vitro, ex vivo or in vivo. In certain embodiments, the compositions are introduced into a live, intact mammal. The mammal may be at any stage of development at the time of delivery, e.g., embryonic, fetal, neonatal, infantile, juvenile or adult. Additionally, targeted cells may be healthy or diseased. In certain embodiments, one or more of the compositions are delivered to a specific tissue (e.g., spleen or lymph node), intra-arterially, intraperitoneally, or intramuscularly. Ex vivo delivery may be performed with homogeneous or heterogenous populations of cells including stem cells, B cell progenitor cells and/or mature B cells.

A kit, comprising one or more of the expression constructs, AAV vectors, B cell and/or pharmaceutical compositions described herein, is also provided. The kit may further comprise nucleic acids encoding nucleases, (e.g. RNA molecules encoding ZFNs, TALENs or Cas and modified Cas proteins, and guide RNAs), or aliquots of the nuclease proteins, cells, instructions for performing the methods of the invention, and the like.

These and other aspects will be readily apparent to the skilled artisan in light of disclosure as a whole.

### BRIEF DESCRIPTION OF THE DRAWINGS

FIG. 1 is a schematic showing an overview of the in vitro B cell thawing and differentiation protocol followed (see Jourdan et al (2009) Blood 114:5173-5181).

FIGS. 2A through 2C are graphs demonstrating the ability of the in vitro differentiated B cells to produce antibodies, including IgM antibodies (FIG. 2A), IgG antibodies (FIG. 2B) and IgA antibodies (FIG. 2C). Samples were treated with cytokines (“+cytokines”) or not, and then the amount of antibody detected by ELISA. Supernatants were collected on days t4, t7 and t10 and total IgM, IgG, and IgA antibody levels were quantified by specific ELISA. Data represents technical duplicates. Error bars represent standard deviation.

FIGS. 3A through 3D are graphs depicting the percent of GFP positive cells following mRNA electroporation into the B cells 0 days (FIG. 3A), 1 day (FIG. 3B), 2 days (FIG. 3C) or 3 days (FIG. 3D) following thaw. CD19+ B cells were electroporated with mRNA on t0, t1, t2 and t3 where t equals days following thaw to determine the optimal time point for mRNA addition. CD19+ positive B cells (2.0E+05 cells) were mixed with GFP mRNA (2 μg) followed by electroporation. Cells were collected 24 hours later and analyzed by flow cytometry to assess GFP levels. Day 2 post thaw (t2) was had the highest levels of GFP and was chosen for further studies. Data represents technical duplicates. Error bars represent standard deviation.

FIGS. 4A and 4B are graphs depicting flow cytometry gating for GFP expression in transduced cells. FIG. 4A defines the areas of the plot associated with side scatter (“SSC”) and forward scatter (“FSC”). FIG. 4B exemplifies the differences between a mock treated set of B cells (left panel) and those electroporated with GFP encoding mRNA (right panel). As can be seen in the right panel of FIG. 4B, the expression of the GFP mRNA results in an increase in GFP generated fluorescence that can is quantifiable following gating.

FIGS. 5A through 5C are graphs depicting genome editing in B cells. Deep sequencing at multiple loci demonstrated robust genome editing using zinc finger nucleases targeting AAVS1, CCR5 and TCRA (TRAC). Percent genome modification was calculated by dividing the insertion and deletion (indels) containing sequence count by the total sequence count. CD19+ B cells (2.0E+5 cells) were mixed with ZFN mRNA (4 μg) followed by electroporation. Cells were collected over time (t=days) following transfection. Data represents technical duplicates. Error bars represent standard deviation. FIG. 5A depicts genome editing at the AAVS1 locus at days 4, 7, and 10. FIG. 5B shows a similar data set at the CCR5 locus while FIG. 5C shows the data at the TCRA (TRAC) locus.

FIGS. 6A through 6C show the effect of a transient cold shock on B cell genome editing. CD19+ B cells (2.0E+5 cells) were mixed with ZFN mRNA (0.75, 1.5, 3 and 6 μg) followed by electroporation. Post-electroporation cells were split into two groups. One group was placed in a 37° C. incubator for 4 days. The second group was placed in a 30° C. overnight, then transferred to a 37° C. incubator for 3 days. Deep sequencing revealed an increase in genome editing (% indels) in cells treated with the transient cold shock.

FIGS. 7A and 7B depict the transduction ability of several AAV serotypes tested for delivery of a CMV promoter-GFP donor into CD19+ B cells. FIG. 7A shows the results for recombinant AAV serotypes 2, 5, 6, 8 and 9 harboring CMV-GFP delivered at vector doses of 2.4E+6, 1.2E+6, 6.0E+5, 3.0E+5 vector genomes (vg)/cell. Data shown are of n=2 biological replicates where cells were analyzed for GFP expression at days 4, 7 and 10 after transduction, and demonstrate that AAV6 readily transduces B cells. Error bars represent standard deviation of technical and biological replicates. FIG. 7B is a schematic depiction of the expression cassette used in experiments shown.

FIG. 8 depicts exemplary AAV expression cassettes used. Exemplary donor cassettes for insertion into the AAVSI, CCR5 and TCRA (TRAC) loci are shown. AAVS1 has a left (“AAVS1-L”) and right (“AAVS1-R”) homology arm consisting of 801 and 568 base pairs in length, respectively. CCR5 has a left (“CCR5-L”) and right (“CCR5-R”) homology arm consisting of 473 and 1431 base pairs in length, respectively. TCRA (“TRAC”) has a left (“TRAC-L”) and right (“TRAC-R”) homology arm consisting of 925 and 989 base pairs in length, respectively. Donors contained either a phosphoglycerate kinase (“PGK”) or B cell specific promoter (“EEK”, comprising the light chain promoter (VKp) preceded by an intronic enhancer (iE^{κ}), a MAR, and a 3 enhancer (3′ E^{κ}); U.S. Pat. No. 8,133,727) followed by GFP-encoding transgene (“GFP”) and a bovine growth hormone polyadenylation signal (“pA”). AAV2 inverted terminal repeats (“ITR”s) were used to enable packaging into AAV capsids.

FIGS. 9A through 9C are graphs showing that a combination of ZFN mRNA and rAAV2/6 vectors promoted high levels of transgene addition at multiple loci. Levels of GFP expression were measured by flow cytometry. B cell cultures were collected over time (t=days) following addition of ZFN mRNA and AAV donor to B cells. AAVS1 (FIG. 9A), CCR5 (FIG. 9B) and TCRA (TRAC, FIG. 9C) loci were evaluated for transgene addition. ZFN:Donor samples show durable GFP expression while Donor only samples show a decrease in GFP expression over time. Below each graph is shown the target site for the nucleases (for example, in FIG. 9A, “ZFN: AAVS1”). Also shown is a description of the GFP transgene (for example, in FIG. 9A, “Donor: PGK-AAVS1” indicates that the GFP transgene was driven from the PGK promoter, and that the GFP coding sequence was flanked by homology arms with homology to the nuclease site in the AAVS1 gene). In FIGS. 9A through 9C, the left panel shows results following collection 4 days (t4) after transfection; the middle panel shows results following collection 7 days (t7) after transfection; and the right panel shows results following collection 10 days after transfection.

FIGS. 10A and 10B are graphs showing the percent of targeted integration of the GFP donor at the AAVS1 (FIG. 10A) and CCR5 (FIG. 10B) loci in the CD19+ B cells. Confirmation of target integration (transgene addition) was done by deep sequencing. Percent gene modification was calculated by dividing the integrated target containing sequence count by the total sequence count. B cells were collected over time (t=days) following addition of ZFN mRNA and AAV donor to B cells. AAVSI data represents 3 independent experiments. CCR5 data represents 2 independent experiments. Error bars represent the standard deviation. Below each graph is shown the target site for the nucleases and the configuration of the donor as described above.

FIG. 11 is a graph depicting the results to determine whether homology-driven recombination (HDR) or end capture via non-homologous end joining (NHEJ) are used by the B cells for targeted integration. The table below the graph shows the ZFN specificity and the donor configuration. All donors used the PGK promoter, but only experiment #1 had the donor GFP transgene flanked by homology arms matching the nuclease cut site. Mismatched ZFN and donor homology arm samples show similar expression of GFP as donor only without any added nuclease. The greatest targeted integration occurred when the homology arms matched the nuclease target site, demonstrating that HDR is used for integration in B cells.

FIG. 12 is a graph depicting the comparison of the PGK promoter driving GFP expression with a B cell-specific promoter EEK driving the GFP expression. B cells were mixed with ZFN mRNA (4 μg) targeting the TCRA (TRAC) locus, followed by electroporation. Following electroporation, CD19+ B cells were then transduced with AAV6 containing TRAC homology arms flanking the transgene expression cassette and either a PGK or B cell specific promoter (EEK) driving GFP transgene expression. These were delivered at vector dose of 2.4E+06 vg/cell. The data demonstrate that the use of the B cell specific promoter (EEK) showed a slight increase in GFP expression compared to the PGK promoter.

FIGS. 13A through 13D are graphs depicting the amount of GFP transgene expression in the CD19+ B cells at a range of donor AAV doses. In all cases, the GFP transgene was being integrated into the TCRA (TRAC) locus using TCRA-specific nucleases. Also shown in each graph of the GFP expression results when the transduction was done in the absence of the nucleases. The donor constructs comprised TCRA-specific homology arms and either an EEK promoter as described above or a PGK promoter. The range of AAV used included 3.0E+05 vg/cell (FIG. 13D), 6.0E+05 vg/cell (FIG. 13C), 1.2E+06 vg/cell (FIG. 13B) and 2.4E+06 vg/cell (FIG. 13A). CD19+ positive B cells were mixed with ZFN encoding mRNA (4 μg) targeting the TCRA locus, followed by electroporation. After electroporation, CD19+ B cells were transduced with AAV containing TCRA homology arms, with either a PGK or B cell specific (EEK) promoter driving GFP expression. These were delivered at vector doses of 2.4E+06, 1.2E+06, 6.0E+05 and 3.0E+05 vg/cell. The percentage of GFP expression driven by the PGK promoter decreased as dose decreased whereas the B cell specific promoter maintained GFP expression over an 8-fold dilution. There is almost a 5-fold difference at 3.0E+05 vg/cell between the two promoters.

FIGS. 14A through 14C are graphs depicting impact on antibody production following the genome editing manipulations demonstrating no major loss of IgG in vitro production as measured by ELISA as a result of the manipulations. Total secreted IgG levels are similar independent of treatment over the course of the experiment (representing combined secreted IgG levels on days 4, 7 and 10) indicating electroporation and transduction do not negatively impact IgG production. Addition of cytokines is essential for IgG production. CD19+ B cells were treated with either AAVS1 specific ZFN (FIG. 14A), CCR5 specific ZFN (FIG. 14B) or TCRA (TRAC) specific ZFN (FIG. 14C). The various conditions used for each data set included the specific ZFN paired with the GFP transgene with the matching homology arms (“ZFN:Donor”), GFP transgene and homology arms (“Donor”), specific ZFN alone (“ZFN”), CD19+ B cells treated in the BTX device with buffer only (“BTX cells”), untreated CD19+ B cells plus cytokines (“B cells”) and untreated CD19+ B cell with no cytokines (“B cells-Cytos”). The ZFN:Donor, Donor, ZFN, BTX Cells and B cells were all treated with cytokines.

FIGS. 15A through 15C are graphs depicting impact on antibody production following the genome editing manipulations demonstrating no major loss of IgM in vitro production as measured by ELISA. CD19+ B cells were treated with either AAVS1 specific ZFN (FIG. 15A), CCR5 specific ZFN (FIG. 15B) or TCRA (TRAC) specific ZFN (FIG. 15C). Samples are as described above in FIG. 14.

FIG. 16 is a graph depicting IgM production from differentiated CD19+ B cells treated with cytokines from a single human donor. The CD19+ B cells were subject to treatment with AAV2, 5, 6, 8 or 9 virus. In the presence of added cytokines, IgM production, as measured by ELISA, was ‘boosted’ when treated with the AAV2 only. Prevalence of antibodies to wild-type AAV in the human population is robust and has not been associated with disease. Shown here is a potential boost of antibody production due to what would be considered re-infection by AAV.

FIGS. 17A and 17B are illustrations depicting a potential mechanism for the increased IgM expression as a result of the AAV2 ‘boost’. Depicted in the left panel (FIG. 17A) is a simplified scenario of production of antibodies in a B cell following AAV infection. The panel shown on the right (FIG. 17B) is an example denoting how the AAV could be harnessed to function as a booster to increase expression of an inserted transgene driven from an antibody promoter in an engineered B cell.

### DETAILED DESCRIPTION

Disclosed herein are methods and compositions for genetic engineering of a B cell, including knocking out endogenous genes and inserting (stably or episomally) expression cassettes for expression of a transgene. The methods can be carried out in vitro, ex vivo or in vivo and can be used to express any transgene(s) for the treatment and/or prevention of any disease or disorder which can be ameliorated by the provision of one or more of the transgenes.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Recombination” refers to a process of exchange of genetic information between two polynucleotides, including but not limited to, capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms.

In certain methods of the disclosure, one or more targeted nucleases as described herein create a double-stranded break (DSB) in the target sequence (e.g., cellular chromatin) at a predetermined site (e.g., albumin gene). The DSB mediates integration of a construct as described herein. Optionally, the construct has homology to the nucleotide sequence in the region of the break. The expression construct may be physically integrated or, alternatively, the expression cassette is used as a template for repair of the break via homologous recombination, resulting in the introduction of all or part of the nucleotide sequence as in the expression cassette into the cellular chromatin. Thus, a first sequence in cellular chromatin can be altered and, in certain embodiments, can be converted into a sequence present in an expression cassette. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, the exogenous nucleotide sequence (the “expression construct” or “expression cassette” or “vector”) can contain sequences that are homologous, but not identical, to genomic sequences in the region of interest, thereby stimulating homologous recombination to insert a non-identical sequence in the region of interest. Thus, in certain embodiments, portions of the expression cassette sequence that are homologous to sequences in the region of interest exhibit between about 80 to 99% (or any integer therebetween) sequence identity to the genomic sequence that is replaced. In other embodiments, the homology between the expression cassette and genomic sequence is higher than 99%, for example if only 1 nucleotide differs as between the homology regions of the expression cassette and genomic sequences of over 100 contiguous base pairs. In certain cases, a non-homologous portion of the expression cassette can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest.

The term “sequence” refers to a nucleotide sequence of any length, which can be DNA or RNA; can be linear, circular or branched and can be either single-stranded or double stranded. The term “transgene” refers to a nucleotide sequence that is inserted into a genome. A transgene can be of any length, for example between 2 and 100,000,000 nucleotides in length (or any integer value therebetween or thereabove), preferably between about 100 and 100,000 nucleotides in length (or any integer therebetween), more preferably between about 2000 and 20,000 nucleotides in length (or any value therebetween) and even more preferable, between about 5 and 15 kb (or any value therebetween).

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes. The liver specific constructs described herein may be episomally maintained or, alternatively, may be stably integrated into the cell.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, ligases, deubiquitinases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

As used herein, the term “product of an exogenous nucleic acid” includes both polynucleotide and polypeptide products, for example, transcription products (polynucleotides such as RNA) and translation products (polypeptides).

A “fusion” molecule is a molecule in which two or more subunit molecules are linked, preferably covalently. The subunit molecules can be the same chemical type of molecule, or can be different chemical types of molecules. Examples of fusion molecules include, but are not limited to, fusion proteins (for example, a fusion between a protein DNA-binding domain and a cleavage domain), fusions between a polynucleotide DNA-binding domain (e.g., sgRNA) operatively associated with a cleavage domain, and fusion nucleic acids (for example, a nucleic acid encoding the fusion protein).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, TALE or CRISPR/Cas system as described herein. Thus, gene inactivation may be partial or complete. A “genetically modified” cell includes cells with any change to the genetic material in the cell, including but not limited to episomal and/or genomic modifications. Non-limiting examples of genetic modifications includes insertions and/or deletions (for example episomal and/or targeted integration of one or more transgenes, RNAs or non-coding sequences) and/or mutations (for example point mutations, substitutions, etc.) that alter protein expression within the cell).

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., B-cells), including stem cells (pluripotent and multipotent).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the B-domain deleted human Factor VIII is a functional fragment of the full-length Factor VIII protein.

A polynucleotide “vector” or “construct” is capable of transferring gene sequences to target cells. Typically, “vector construct,” “expression vector,” “expression construct,” “expression cassette,” and “gene transfer vector,” mean any nucleic acid construct capable of directing the expression of a gene of interest and which can transfer gene sequences to target cells. Thus, the term includes cloning, and expression vehicles, as well as integrating vectors.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the expression cassettes of the invention can be administered. Subjects of the present invention include those with a disorder.

### B Cell Expression Constructs

Described herein are expression cassettes (constructs) for use in directing expression of a transgene in a B cell (including plasmablasts and plasma cells), including in vivo following administration of the expression cassette(s) to the subject (e.g., intravenous delivery). The expression construct may be maintained episomally and drive expression of the transgene extrachromosomally or, alternatively, the expression construct may be integrated into the genome of a B cell, for example by nuclease-mediated targeted integration.

Any suitable promoter sequence can be used in the expression cassettes of the invention. In certain embodiments, the promoter is a constitutive promoter. In other embodiments, the promoter is inducible and/or is a B cell specific promoter. Promoterless constructs in which the transgene is driven by an endogenous B cell promoter are also contemplated for genetic modification of cells as described herein.

As will be apparent, any transgene can be used in the constructs described herein. Furthermore, the individual expression construct components (promoter, enhancer, insulator, intron, transgene, etc.) of the constructs described herein may be present or not, and may mixed and matched in any combination.

The constructs described herein may be contained within any viral or non-viral vector. The constructs may be maintained episomally or may be integrated into the genome of the cell (e.g., via nuclease-mediated targeted integration).

Non-viral vectors include DNA or RNA plasmids, DNA MCs, naked nucleic acid, and nucleic acid complexed with a delivery vehicle such as a liposome, lipid nanoparticle, nanoparticle or poloxamer. Viral vectors that may be used to carry the expression cassettes described herein include, but are not limited to, retroviral, lentivirus, adenoviral, adeno-associated viral vectors, vaccinia and herpes simplex virus vectors. Integration in the host genome is possible with the retrovirus, lentivirus, and adeno-associated virus gene transfer methods, and as described herein may be facilitated by nuclease-mediated integration.

In certain preferred embodiments, the constructs are included in an adeno-associated virus (“AAV”) vector or vector system that may be maintained episomally or integrated into the genome of a B cell (e.g., via nuclease-mediated targeted integration). Construction of recombinant AAV vectors is in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

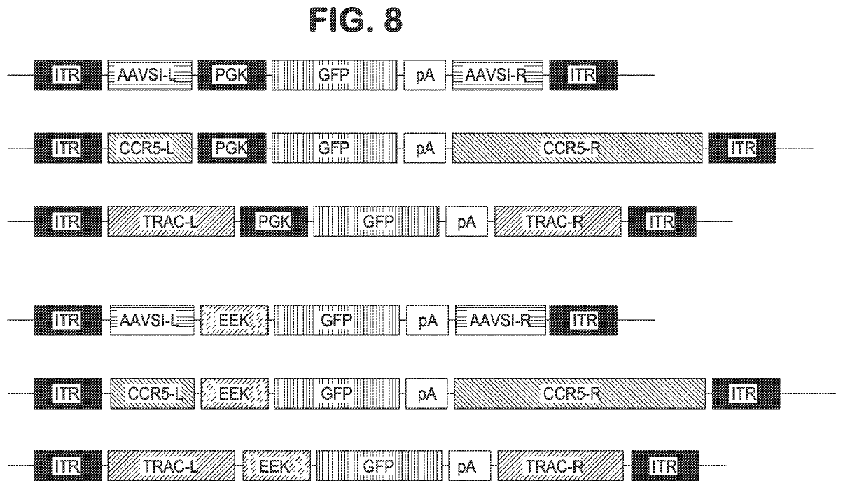

Thus, in certain embodiments, the expression construct is carried on an AAV construct and further comprises 5′ and 3′ ITRs flanking the expression constructs elements (e.g., enhancer, promoter, optional intron, transgene, etc.) as described herein. Optionally, spacer molecules are also included between one or more of the components of the expression construct, for example, between the 5′ ITR and the enhancer and/or between the polyadenylation signal and the 3′ ITR. The spacers may function as homology arms to facilitate recombination into a safe-harbor locus (e.g. albumin). In certain embodiments, the construct is a construct as shown in FIG. 8.

In certain embodiments, the AAV vectors as described herein can be derived from any AAV. In certain embodiments, the AAV vector is derived from the defective and nonpathogenic parvovirus adeno-associated type 2 virus. All such vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV2, AAV3, AAV4, AAV5, AAV6, AAV7, AAV8, AAV9 and AAVrh.10 and any novel AAV serotype can also be used in accordance with the present invention. Especially preferred are AAV6 serotypes. In some embodiments, chimeric AAV is used where the viral origins of the ITR sequences of the viral nucleic acid are heterologous to the viral origin of the capsid sequences. Non-limiting examples include chimeric virus with ITR derived from AAV2 and capsids derived from AAV5, AAV6, AAV8 or AAV9 (i.e. AAV2/5, AAV2/6, AAV2/8 and AAV2/9, respectively).

Retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

The constructs described herein may also be incorporated into an adenoviral vector system. Adenoviral based vectors are capable of very high transduction efficiency in many cell types and do not require cell division. With such vectors, high titer and high levels of expression have been obtained. This vector can be produced in large quantities in a relatively simple system.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Replication-deficient recombinant adenoviral vectors (Ad) can also be used with the polynucleotides described herein. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include HEK293 and Sf9 cells, which can be used to package AAV and adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In some embodiments, AAV is produced using a baculovirus expression system (see e.g. U.S. Pat. Nos. 6,723,551 and 7,271,002).

Purification of AAV particles from a 293 or baculovirus system typically involves growth of the cells which produce the virus, followed by collection of the viral particles from the cell supernatant or lysing the cells and collecting the virus from the crude lysate. AAV is then purified by methods known in the art including ion exchange chromatography (e.g. see U.S. Pat. Nos. 7,419,817 and 6,989,264), ion exchange chromatography and CsCl density centrifugation (e.g. PCT publication WO2011094198A10), immunoaffinity chromatography (e.g. WO2016128408) or purification using AVB Sepharose (e.g. GE Healthcare Life Sciences).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

The polynucleotides described herein may include one or more non-natural bases and/or backbones. In particular, an expression cassette as described herein may include methylated cytosines to achieve a state of transcriptional quiescence in a region of interest.

Furthermore, the expression constructs as described herein may also include additional transcriptional or translational regulatory or other sequences, for example, Kozak sequences, additional promoters, enhancers, insulators, introns, internal ribosome entry sites, sequences encoding 2A peptides, furin cleavage sites and/or polyadenylation signals. Further, the control elements of the genes of interest can be operably linked to reporter genes to create chimeric genes (e.g., reporter expression cassettes).

### Modifications

Described herein are genetically modified B cells comprising one or more of the following modifications: (a) the provision in the cell of one or more transgenes (episomal and/or integrated in any combinations); (b) insertions and/or deletions in one or more genes which modify (i) B cell receptor genes, and/or (ii) cellular interactions in Germinal Centers; and/or (c) modifications (mutations) that inhibit suppression of any B cell function associated with pathogen infection or cancer regulation. Genetically modified B cells as described herein may also be descended from HSCs comprising one or more of these genetic modifications.

In certain embodiments, the constructs described herein can be used for B cell expression of any transgene(s). One or more transgenes may be expressed episomally in the modified B cells and/or following nuclease-mediated targeted integration of one or more of the transgenes. Exemplary transgenes (also referred to as genes of interest and/or exogenous sequences) include, but are not limited to any polypeptide coding sequence (e.g., cDNAs), promoter sequences, enhancer sequences, epitope tags, marker genes, cleavage enzyme recognition sites and/or various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence.

In a preferred embodiment, the transgene comprises a polynucleotide encoding any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the above. The coding sequences may be, for example, cDNAs.

In certain embodiments, the transgene(s) encode(s) functional versions of proteins lacking of deficient in any genetic disease, including but not limited to, lysosomal storage disorders (e.g., Gaucher, Fabry, Hunter, Hurler, Neimann-Pick, etc.), metabolic disorders, and/or blood disorders such as hemophilias and hemoglobinopathies, etc. See, e.g., U.S. Publication No. 20140017212 and 20140093913; U.S. Pat. Nos. 9,255,250 and 9,175,280.

For example, the transgene may comprise a sequence encoding a polypeptide that is lacking or non-functional in the subject having a genetic disease, including but not limited to any of the following genetic diseases: achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasiaossificans progressive, fragile X syndrome, galactosemis, Gaucher's disease, generalized gangliosidoses (e.g., GM1), hemochromatosis, the hemoglobin C mutation in the 6^{th} codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesisimperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240), acquired immunodeficiencies, lysosomal storage diseases (e.g., Gaucher's disease, GM1, Fabry disease and Tay-Sachs disease), mucopolysaccahidosis (e.g. Hunter disease, Hurler disease), hemoglobinopathies (e.g., sickle cell diseases, HbC, α-thalassemia, β-thalassemia) and hemophilias.

Non-limiting examples of proteins (including functional fragments thereof such as truncated versions) that may be expressed as described herein include fibrinogen, prothrombin, tissue factor, Factor V, Factor VII, Factor VIII, Factor IX, Factor X, Factor XI, Factor XII (Hageman factor), Factor XIII (fibrin-stabilizing factor), von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, glucocerebrosidase (GBA), α-galactosidase A (GLA), iduronate sulfatase (IDS), iduronidase (IDUA), acid sphingomyelinase (SMPD1), MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (ornithine transcarbamylase), ASS (argininosuccinic acid synthetase), ASL (argininosuccinase acid lyase) and/or ARG1 (arginase), and/or a solute carrier family 25 (SLC25A13, an aspartate/glutamate carrier) protein, a UGT1A1 or UDP glucuronsyltransferase polypeptide A1, a fumarylacetoacetate hydrolyase (FAH), an alanine-glyoxylate aminotransferase (AGXT) protein, a glyoxylate reductase/hydroxypyruvate reductase (GRHPR) protein, a transthyretin gene (TTR) protein, an ATP7B protein, a phenylalanine hydroxylase (PAH) protein, a lipoprotein lyase (LPL) protein, an engineered nuclease, an engineered transcription factor and/or a therapeutic single chain antibody.

In other embodiments, the engineered B-cells described herein include one or more transgenes encoding one or more antibodies that are engineered molecules designed to target immune cells via specific molecular targets expressed on cell surfaces. In some embodiments, the engineered B-cells express antibodies designed to target endogenous B cells. These antibodies may induce antibody meditated killing (e.g., through ADCC or complement mediated killing) of B cells or other immune cells involved in attenuating an immune response.

B cells as described herein can be genetically modified to produce one or more antibodies that are specific for B cells producing undesirable antibodies. Non-limiting examples of B cells producing undesirable antibodies include B cells producing antibodies against proteins administered in ERT (clotting factors such as F8, F9, etc. in hemophilia patients, and/or proteins lacking or deficient in lysosomal storage disorders). The antibody-encoding constructs are introduced into the B-cell precursor or B-cell ex vivo, such that when the cell is re-introduced into the patient the antibody producing B-cells specifically target cells (B cells) producing the protein (e.g. antibody) bound by the engineered antibody. In certain embodiments, the engineered antibody is specific for antibodies directed against a therapeutic protein supplied exogenously (via ERT and/or gene therapy) such that the antibodies against the therapeutic proteins are neutralized. Thus, the compositions and methods described herein include engineered B-cells that produce antibodies that specifically target antibodies (e.g., anti-F9 antibodies) produced by in the patient. The engineered B-cells of these compositions and methods may be administered to the subject as mature B-cells, or as precursor cells (such as HSCs or lymphoid progenitor cells) that differentiate in the subject after administration or, alternatively, may be genetically modified in vivo. In still further embodiments, the proteins produced from the transgenes (for example anti-ERT antibodies) of the genetically modified B-cells are isolated an administered to the subject in need thereof, for example a patient in need of antibodies to the anti-ERT antibodies their body has generated.

In still further embodiments, the transgene may be an antibody specific for a B cell that is sensitive to a protein involved in an autoimmune disease. The term “autoimmune disease” refers to any disease or disorder in which the subject mounts a destructive immune response against its own tissues. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes. Thus, the B cells as described herein can comprise a molecule (e.g., engineered antibody) directed to a B cell population in a subject that is sensitive to (and produces antibodies against) an autoantigen involved in an autoimmune disease, including but not limited to myelin basic protein (MBP), insulin, ANA, joint or muscle proteins, thyroid proteins and the like.

In certain embodiments, the transgene can comprise a marker gene (described above), allowing selection of cells that have undergone targeted integration, and a linked sequence encoding an additional functionality. Non-limiting examples of marker genes include GFP, drug selection marker(s) and the like.

The constructs described herein may also be used for delivery of non-coding transgenes. Sequences encoding antisense RNAs, RNAi, shRNAs and micro RNAs (miRNAs) may also be used for targeted insertions.

In certain embodiments, the transgene includes sequences (e.g., coding sequences, also referred to as transgenes) greater than 1 kb in length, for example between 2 and 200 kb, between 2 and 10 kb (or any value therebetween). The transgene may also include one or more nuclease target sites. The transgene may also comprise one or more homology arms. The homology arms comprise sequences with a high degree of homology to those flanking a nuclease cleavage target site. A homology arm can comprise 50, 100, 200, 500, 1000, 2000 or more nucleotides or any value therebetween.

When integrated (e.g., via nuclease-mediate integration), the transgene may be inserted into an endogenous gene such that all, some or none of the endogenous gene is expressed.

### Nucleases

As noted above, the expression cassettes may be maintained episomally or may be integrated into the genome of the cell. Integration may be random. In certain embodiments, integration of the transgene construct(s) is targeted to a specified gene following cleavage of the target gene by one or more nucleases (e.g., zinc finger nucleases (“ZFNs”), TALENs, TtAgo, CRISPR/Cas nuclease systems, and homing endonucleases) and the construct integrated by either homology directed repair (HDR) or by end capture during non-homologous end joining (NHEJ) driven processes. See, e.g., U.S. Pat. Nos. 9,394,545; 9,150,847; 9,206,404; 9,045,763; 9,005,973; 8,956,828; 8,936,936; 8,945,868; 8,871,905; 8,586,526; 8,563,314; 8,329,986; 8,399,218; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 20100218264; 20120017290; 20110265198; 20130137104; 20130122591; 20130177983 and 20130177960 and 20150056705, the disclosures of which are incorporated by reference in their entireties for all purposes.

Any nuclease can be used for targeted integration of the transgene expression construct.

In certain embodiments, the nuclease comprises a zinc finger nuclease (ZFN), which comprises a zinc finger DNA-binding domain and a cleavage (nuclease) domain. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861.

In other embodiments, the nuclease comprises a TALEN, which comprises a TAL-effector DNA binding domain and a cleavage (nuclease) domain. See, e.g., U.S. Pat. No. 8,586,526 and U.S. Publication No. 20130196373.

In still further embodiments, the nuclease comprises a CRISPR/Cas nuclease system, which includes a single guide RNA for recognition of the target site and one or more cleavage domains. See, e.g., U.S. Patent Publication No. 20150056705. In some embodiments, the CRISPR-Cpfl system is used (see Fagerlund et al, (2015) Genom Bio 16:251). It is understood that the term “CRISPR/Cas” system refers to both CRISPR/Cas and CRISPR/Cfpl systems.

The cleavage domains of the nucleases may be wild-type or mutated, including non-naturally occurring (engineered) cleavage domains that form obligate heterodimers. See, e.g., U.S. Pat. Nos. 8,623,618; 7,888,121; 7,914,796; and 8,034,598 and U.S. Publication No. 20110201055.

The nuclease(s) may make one or more double-stranded and/or single-stranded cuts in the target site. In certain embodiments, the nuclease comprises a catalytically inactive cleavage domain (e.g., FokI and/or Cas protein). See, e.g., U.S. Pat. Nos. 9,200,266; 8,703,489 and Guillinger et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffery et al. (2016) Nucleic Acids Res. 44(2):el11. doi: 10.1093/nar/gkv878. Epub 2015 Oct. 19.

In certain embodiments, the nuclease cleaves a safe harbor gene (e.g., CCR5, Rosa, albumin, AAVS1, TCRA, TCRB, etc. See, e.g., U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20060063231; 20080159996; 201000218264; 20120017290; 20110265198; 20130137104; 20130122591; 20130177983 and 20130177960. In preferred embodiments, the nuclease cleaves an endogenous albumin gene such that the expression cassette is integrated into the endogenous albumin locus of a liver cell. Albumin-specific nucleases are described, for example, in U.S. Pat. No. 9,150,847; and U.S. Publication Nos. 20130177983 and 20150056705.

### Delivery

The constructs described herein (and/or nucleases) may be delivered in vivo by any suitable means into any cell type, preferably to the spleen or secondary lymph nodes. Similarly, when used in combination with nucleases for targeted integration, the nucleases may be delivered in polynucleotide and/or protein form, for example using non-viral vector(s), viral vectors(s) and/or in RNA form, e.g., as mRNA.

Methods of non-viral delivery of nucleic acids include electroporation, lipofection, microinjection, biolistics, virosomes, liposomes, lipid nanoparticles, immunoliposomes, other nanoparticle, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids. Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336).

In preferred embodiments, the expression constructs are AAV vectors. The optional nucleases may be administered in mRNA form or using one or more viral vectors (AAV, Ad, etc.). Administration can be by any means in which the polynucleotides are delivered to the desired target cells. Both in vivo and ex vivo methods are contemplated. Intravenous injection in a peripheral blood vessel is a preferred method of administration. Other in vivo administration modes include, for example, direct injection into tissues comprising B cells including lymph nodes, bone marrow, plasma, lymphatic system and the spleen.

In systems involving delivery of more than one polynucleotides (e.g., construct as described herein and nuclease in polynucleotide form), the two or more polynucleotide(s) are delivered using one or more of the same and/or different vectors. For example, the nuclease in polynucleotide form may be delivered in mRNA form and the B-cell-specific constructs as described herein may be delivered via other modalities such as viral vectors (e.g., AAV), minicircle DNA, plasmid DNA, linear DNA, liposomes, lipid nanoparticles, nanoparticles and the like.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

The effective amount of expression cassette (and optional nuclease(s), and/or modified cells) to be administered will vary from patient to patient. Accordingly, effective amounts are best determined by the physician administering the compositions (e.g., cells) and appropriate dosages can be determined readily by one of ordinary skill in the art. Analysis of the serum, plasma or other tissue levels of the therapeutic polypeptide and comparison to the initial level prior to administration can determine whether the amount being administered is too low, within the right range or too high. Suitable regimes for initial and subsequent administrations are also variable, but are typified by an initial administration followed by subsequent administrations if necessary. Subsequent administrations may be administered at variable intervals, ranging from daily to annually to every several years. One of skill in the art will appreciate that appropriate immunosuppressive techniques may be recommended to avoid inhibition or blockage of transduction by immunosuppression of the delivery vectors, see e.g., Vilquin et al., (1995) Human Gene Ther., 6:1391-1401.

Formulations for both ex vivo and in vivo administrations include suspensions (e.g., of genetically modified cells, liposomes, lipid nanoparticles or nanoparticles) in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

### Applications

The methods and compositions disclosed herein are for providing therapies for any disease by provision of a transgene that expresses a product that is lacking or deficient in the disease or otherwise treats or prevents the disease. The cell may be modified in vivo or may be modified ex vivo and subsequently administered to a subject. Thus, the methods and compositions provide for the treatment and/or prevention of such genetic diseases. In addition, the methods and compositions disclosed herein allow for modification of B cells such that these cells exhibit modified toxicity, antibody production and/or processing characteristics.

The following Examples include exemplary embodiments of the present disclosure in which the optionally used nuclease comprises a zinc finger nuclease (ZFN). It will be appreciated that this is for purposes of exemplification only and that other nucleases can be used, for example TALENs, CRISPR/Cas systems, homing endonucleases (meganucleases) with engineered DNA-binding domains and/or fusions of naturally occurring of engineered homing endonucleases (meganucleases) DNA-binding domains and heterologous cleavage domains and/or fusions of meganucleases and TALE proteins. In addition, it will be appreciated that expression constructs as described herein can be carried on other vectors (other than AAV) to produce the same results in the treatment and/or prevention of disorders caused by deficient protein production.

### EXAMPLES

### Example 1: Methods

### Cell Culture

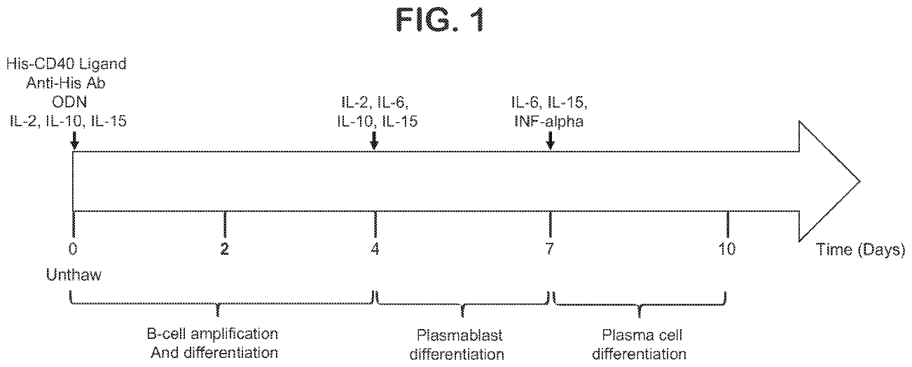

Frozen human peripheral blood CD19+ B cells were purchased from STEMCELL Technologies (Vancouver, Canada). An in vitro B cell differentiation culture system (see FIG. 1) has been described previously (Jourdan et al, ibid). All cultures were performed in Iscove's Modified Dulbecco's Medium (Corning, Corning, N.Y.) and 10% fetal bovine serum (VWR, Radnor, Pa.).

Cells were cultured in a 24-well plate at a density of 2.0E+5 cells per well in 0.5 mL of culture media. Cells were thawed and cultured for 4 days in B cell Activation Media containing Anti-His Ab (5 μg/mL), ODN (10 μg/mL), sCD40L (50 ng/mL), IL-2 (10 ng/mL), IL-10 (50 ng/mL), and IL-15 (10 ng/mL). At day 4 of culture, cells were harvested, supernatants were collected, cells were washed with DPBS and then transferred to Plasma Blast (PB) Generation Media containing IL-2 (10 ng/mL), IL-6 (40-50 ng/mL), IL-10 (50 ng/mL), and IL-15 (10 ng/mL). At day 7 of culture, cells were harvested, supernatants were collected, cells were washed DPBS and then transferred to Plasma Cell (PC) Generation Media containing IL-6 (40-50 ng/mL), IL-15 (10 ng/mL), IFN-α (500 U/mL). At day 10 of culture, cells were harvested and supernatants collected.

### B Cell Gene Modification

### ZFN Reagents:

ZFNs were designed to target TCRA (TRAC, SBS53909 and SBS53885, see U.S. Patent publication No. US-2017-0211075-A1), CCR5 (SBS8266 and SBS8196, see U.S. Pat. No. 7,925,921) and AAVS1 (SBS30035 and SBS30054, see U.S. Pat. No. 8,110,379). The CCR5 and AAVS1 ZFN coding sequences were cloned into a modified version of plasmid pGEM4Z (Promega, Madison, Wis.) containing a sequence of 64 adenines 3′ of the inserted gene sequence (Boczkowski et al (2000) Canc Res 60:1028-1034), which was linearized by SpeI digestion to generate templates for mRNA synthesis. TRAC ZFN mRNA was produced from linear DNA templates (one for each ZFN) via PCR amplification of ZFN-encoding sequence with Accuprime PFX DNA Polymerase Kit (Invitrogen, Carlsbad, Calif.). PCR products were used as templates for mRNA synthesis. mRNA was prepared using the mMESSAGE mMACHINE T7 ULTRA Kit (Life Technologies, Carlsbad, Calif.) per manufacturer's protocol.

Briefly, 1.0 μg of DNA encoding the ZFN was used as template for mRNA synthesis, incubated at 37° C. for two hours in supplied buffer, followed by DNAse digestion supplied with kit. The in vitro poly-A tailing reaction was not performed because a poly-T tail was incorporated on the DNA template during PCR generation of the TRAC template. The AAVS1 and CCR5 templates contain a poly-T template in the vector. mRNA was then purified using the RNeasy Mini Kit (Qiagen, Carlsbad, Calif.) per the manufacturer's protocol and quantified on the Nanodrop 8000 (ThermoScientific, Waltham, Mass.). The primers used for the mRNA templates were

Forward Primer:
(SEQ ID NO: 1)
5′ GCAGAGCTCTCTGGCTAACTAGAG
and
Reverse Primer:
(SEQ ID NO: 2)
5′ TTTTTTTTTTTTTTTTTTTTTTTTTTTTTTTTTTTTTTTTTTTTT
TTTTTTTTTTTTTTTCTGGCAACTAGAAGGCACAG.

### AAV Vectors:

All AAV vectors were produced at Sangamo Therapeutics as described below. AAV donor templates for AAVS1, CCR5 and TRAC contained homology arms to their target loci. AAVS1 had a left and right homology arm of 801 and 568 base pairs in length, respectively. CCR5 had a left and right homology arm of 473 and 1431 base pairs in length, respectively. TRAC had a left and right homology arm of 925 and 989 base pairs in length, respectively. A GFP expression cassette comprising a promoter, a GFP sequence and a human growth hormone polyadenylation signal (hGHpA) was cloned in between the right and left homology arms. The promoter was either a phosphoglycerate kinase (PGK) or B cell specific (EEK) promoter. The B cell specific (EEK) promoter consisted of a 3′-enhancer, a MAR, and an intronic enhancer upstream of human κ light chain promoter (Luo et al (2009) Blood 113:1422-1431). The TRAC donor template was cloned into a pAAV vector. The AAVS1 and CCR5 donors templates were cloned into a customized plasmid pRS 165 (Lombardo et al (2011) Nat Methods 8:861-869; Wang et al (2012) Genome Res 22:1316-1326) derived from pAAV-MCS (Agilent Technologies, Santa Clara, Calif.). AAV2 inverted terminal repeats (ITRs) were used to enable packaging as AAV vectors using the triple-transfection method (Xiao and Samulski (1998) J. Virol 72:2224-2232). Briefly, HEK 293 cells were plated in 10-layer CellSTACK chambers (Corning, Acton, Mass.), grown for 3 days to a density of 80%, then transfected using the calcium phosphate method with an AAV helper plasmid expressing AAV2 Rep and serotype specific Cap genes, an adenovirus helper plasmid, and an ITR-containing donor vector plasmid. After 3 days the cells were lysed by three rounds of freeze/thaw, and cell debris removed by centrifugation. AAV vectors were precipitated from the lysates using polyethylene glycol, and purified by ultracentrifugation overnight on a cesium chloride gradient. Vectors were formulated by dialysis and filter sterilized.

### IgM, IgG, IgA ELISA:

Supernatants were collected at the end of B cell activation, Plasma Blast Generation, and Plasma Cell Generation culture steps. IgM, IgG, IgA were assayed using commercial enzyme-linked immunosorbent assay (ELISA) kits (Bethyl Laboratories; Montgomery, Tex.) according to the manufacture's protocol. Briefly, supernatant was added to the plate, incubated with rocking at room temperature for one hour, followed by washing four times with buffer provided in the kit. Detecting antibody provided with the kit was added and incubated for 1 hour at room temperature, followed by washing four times with wash buffer provided in the kit. Horseradish peroxidase (HRP) provided with the kit was added and incubated for 30 minutes at room temperature, followed by washing four times with buffer provided in the kit. Tetramethylbenzidine (TMB) substrate provided with kit was added and allowed to develop for 30 minutes. The reaction was stopped with Stop Solution provided with the kit and absorbance read at 450 nM using a plate reader.

### Example 2: Antibody Production in In Vitro Cultured B Cells

The CD19+ B cells were thawed and cultured as described above and illustrated in FIG. 1. Culture supernatants were collected on days t4, t7 and t10 and total IgM, IgG and IgA were detected by ELISA as described above.

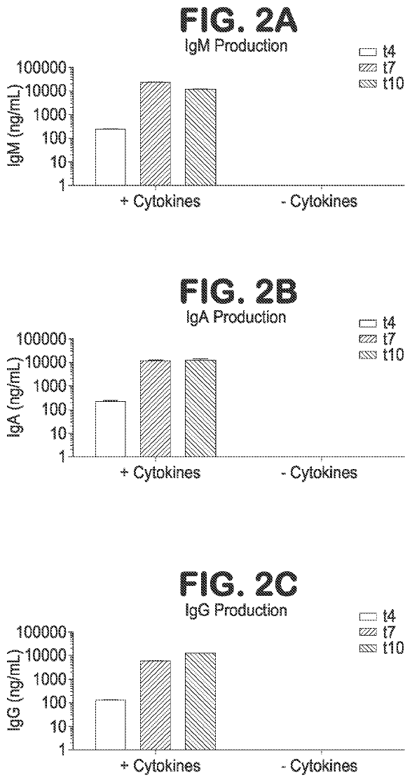

The results (FIG. 2A-2C) demonstrated that the cells are responsive to cytokine stimulation of antibody production and are producing antibodies as would be expected.

### Example 3: mRNA Electroporation

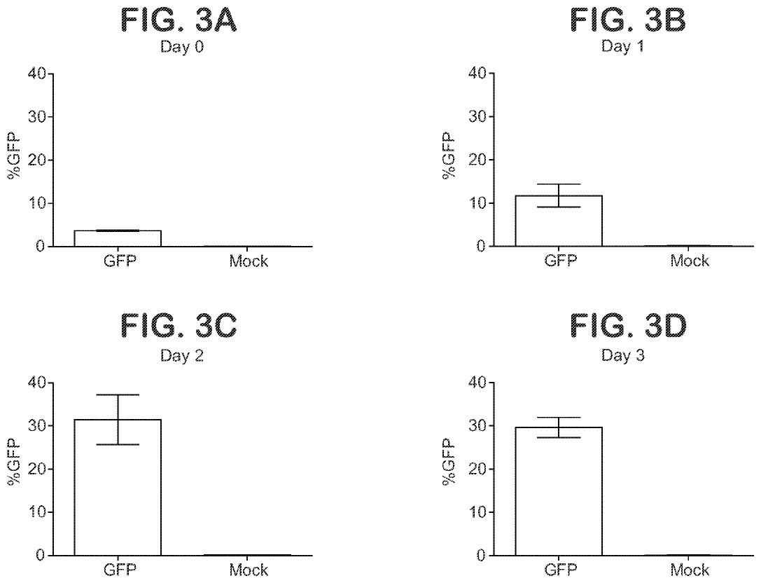

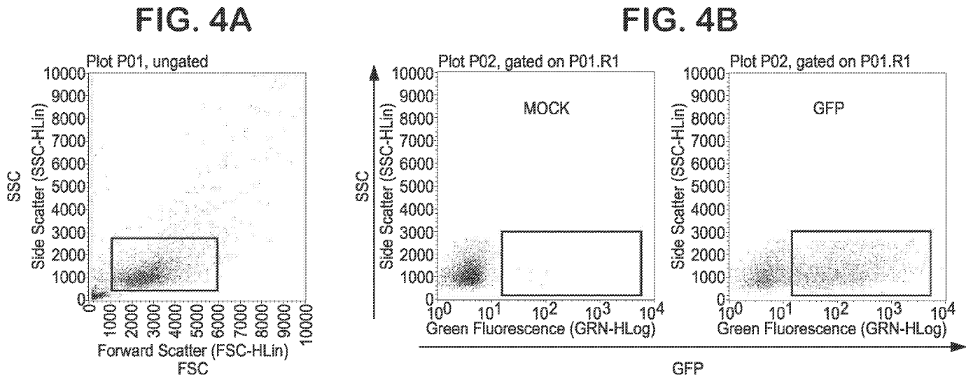

The cultured B cells were treated with mRNAs encoding a transgene (GFP) to determine the best time frame for introduction of the mRNA. The CD19+ B cells (2.0E+05 cells) were electroporated with 2 μg GFP mRNA at days t0, t1, t2 or t3, where t0 is the day the cells were thawed (FIG. 1). The electroporated cells were analyzed by FACs analysis where the gating was performed as shown in FIG. 4. The results (FIGS. 3A-3D) demonstrated that electroporation at day t2 resulted in the highest GFP expression so this time frame was chosen for the follow-on studies.

### Example 4: Nuclease Cleavage of the Cultured B Cells

ZFNs specific for three loci, AAVS1, CCR5 and TCRA were used to cleave their targets in the cultured B cells. CD19+ B cells were thawed and cultured for 2 days in B cell Activation Media. The cells were washed 2 times with DPBS then resuspended in BTXpress high performance electroporation solution (Harvard Apparatus, Holliston, Mass.) to a final density of 2.0E+6 cells/mL. Cells (100 μL) and electroporation solution were mixed with ZFN mRNA (4 μg) followed by electroporation in a BTX ECM830 Square Wave electroporator (Harvard Apparatus) in a MOS 96 multi-well Electroporation Plate 2 mm (Harvard Apparatus). Following electroporation cells were transferred to B cell Activation Media in a 24 well plate for two days. After 2 days, cells were harvested, supernatants were collected and cells were washed with DPBS then transferred to Plasma Blast Generation Media. After 3 days, cells were harvested, supernatants were collected and cells were washed with DPBS then transferred to Plasma Cells Generation Media. Cells were collected for genomic DNA (gDNA) isolation at days t4, t7 and t10 for ZFN activity analysis by deep sequencing. In brief, CD19+ B cells (2.0E+5 cells) were mixed with ZFN mRNA (4 μg) followed by electroporation.

To measure ZFN activity at the TCRA (TRAC), CCR5, and AAVS1 loci, DNA was isolated by QIAamp DNA mini Kit (Qiagen, Carlsbad, Calif.) per the manufacturer's instructions. One hundred nanograms of genomic DNA (gDNA) was used. A two-step PCR for AAVS1 and TRAC loci was then carried out using Phusion® Hot Start Flex Polymerase (New England Biolabs, Ipswich, Mass.). A three-step PCR was used for CCR5 loci. Illumina deep sequencing measured indels at each loci. The primers used for each locus are shown below:

AAVS1 Primers:
AAVS1 Forward:
(SEQ ID NO: 3)
GACGTGTGCTCTTCCGATCTNNNNCCGGTTAATGTGGCTCTGGT
AAVS1 Reverse:
(SEQ ID NO: 4)
ACACGACGCTCTTCCGATCTNNNNGACTAGGAAGGAGGAGGCCT.
The AAVS1 amplicon was:
(SEQ ID NO: 5)
5′NNNNGACTAGGAAGGAGGAGGCCTAAGGATGGGGCTTTTCTGTCAC
CAATCCTGTCCCTAGTGGCCCCACTGTGGGGTGGAGGGGACAGATAAA
AGTACCCAGAACCAGAGCCACATTAACCGGNNNN.
CCR5 Primers:
CCR5 Forward 1:
(SEQ ID NO: 6)
CTGTGCTTCAAGGTCCTTGTCTGC,
CCR5 Reverse 1:
(SEQ ID NO: 7)
CTCTGTCTCCTTCTACAGCCAAGC,
CCR5 Forward 2:
(SEQ ID NO: 8)
CTGCCTCATAAGGTTGCCCTAAG,
CCR5 Reverse 2:
(SEQ ID NO: 9)
CCAGCAATAGATGATCCAACTCAAATTCC,
CCR5 Forward 3:
(SEQ ID NO: 10)
ACACGACGCTCTTCCGATCTNNNNNGCCAGGTTGAGCAGGTAGATG,
CCR5 Reverse 3:
(SEQ ID NO: 11)
AGACGTGTGCTCTTCCGATCTGCTCTACTCACTGGTGTTCATCTTT.
The CCR5 amplicon was:
(SEQ ID NO: 12)
5′NNNNNGCCAGGTTGAGCAGGTAGATGTCAGTCATGCTCTTCAGCCT
TTTGCAGTTTATCAGGATGAGGATGACCAGCATGTTGCCCACAAAACC
AAAGATGAACACCAGTGAGTAGAGC.
TCRA (TRAC) primers:
TCRA Forward:
(SEQ ID NO: 13)
5′ACACGACGCTCTTCCGATCTNNNNCCTCTTGGTTTTACAGATACGA
AC
TCRA Reverse:
(SEQ ID NO: 14)
5′GACGTGTGCTCTTCCGATCTCTCACCTCAGCTGGACCAC
The TCRA amplicon was:
(SEQ ID NO: 15)
5′NNNNCCTCTTGGTTTTACAGATACGAACCTAAACTTTCAAAACCTG
TCAGTGATTGGGTTCCGAATCCTCCTCCTGAAAGTGGCCGGGTTTAAT
CTGCTCATGACGCTGCGGCTGTGGTCCAGCTGAGGTGAG.

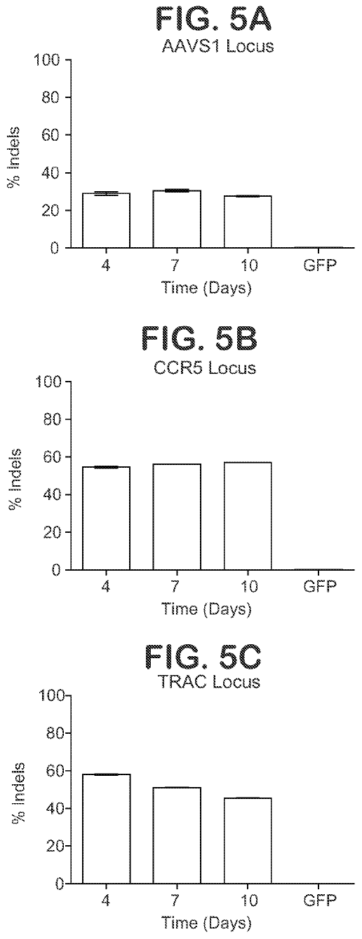

The results of these studies are shown in FIGS. 5A-5C and demonstrate that the nucleases were active in the cultured B cells using these methods, and that greater than 80% modification was achieved at multiple loci.

The experiments were also done testing the impact of a transient (overnight) cold shock (see U.S. Pat. No. 8,772,008) on the cleavage activity of the nucleases. In these studies, a range of input mRNA quantities were used from 0.75 to 6 μg. After electroporation, the cultures were divided and one portion was placed in a 37° C. incubator for 4 days. The second group was placed in a 30° C. overnight, then transferred to a 37° C. incubator for 3 days. Deep sequencing was performed to measure the % indels detected as a result of the nuclease cleavage.

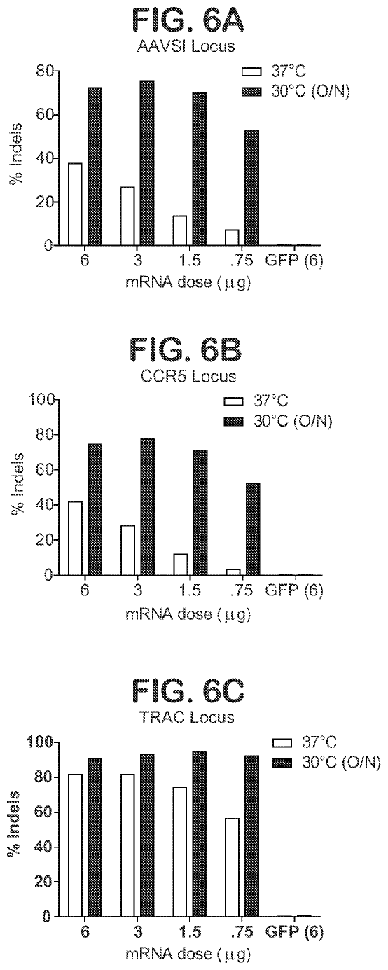

The results (as shown in FIGS. 6A-6C) demonstrated that the cold shock procedure increased overall cleavage activity.

### Example 5: B Cell AAV Serotype Transduction Comparison

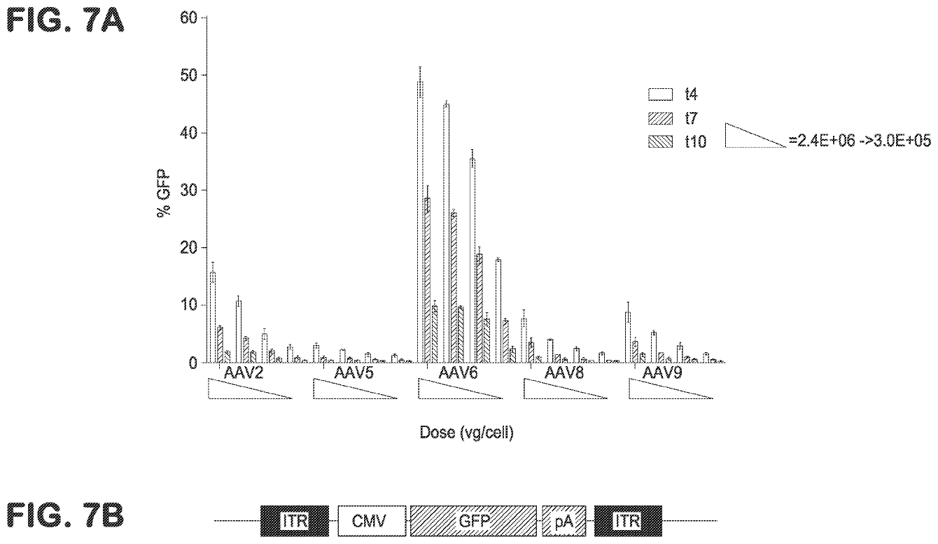

AAV virus comprising a transgene (GFP) expression cassette were used to compare the ability of different AAV serotypes to transduce the cultured B cells. In brief, cells were thawed and cultured for 2 days in B cell Activation Media in a 24-well plate at a density of 2.0E+5 cells/well. Cells were collected, counted and then plated in a 24-well plate at a density of 2.0E+5 cells/well. B cells were transduced with AAV serotypes 2, 5, 6, 8 and 9 at vector doses of 2.4E+6, 1.2E+6, 6.0E+5, 3.0E+5 vector genomes (vg)/cell. AAV vector genomes contained CMV promoter-driven eGFP expression cassette and inverted terminal repeats (ITRs), see FIG. 7B. AAV vectors were produced at Sangamo Therapeutics. Cell culture (25 μL from the 500 μL in a single well of a 24-well plate) was collected and mixed with DPBS (175 μL) at days t4, t7 and t10 and analyzed for GFP expression using a Guava EasyCyte 5HT (EMD Millipore, Billerica, Mass., USA). The data was analyzed using InCyte version 2.5 (EMD Millipore).

The results (FIG. 7A) demonstrated that AAV6 was the most efficient AAV serotype at transducing the cultured B cells during the differentiation process to plasmablasts and plasma cells.

### Example 6: Nuclease Driven Targeted Integration

The nucleases described above were then used in combination with a transgene donor (GFP, proteins lacking or deficient in a subject and/or therapeutic antibodies of interest) to test the ability of the system to support targeted integration of a donor into the genome. Several exemplary donors were made with GFP (FIG. 8), comprising a GFP transgene flanked by homology arms where the arms had homology to the region surrounding either the AAVS1, CCR5 or TCRA cleavage target. In addition, two different promoters, either PGK or EEK were tested.

CD19+ B cells were thawed and cultured for 2 days in B cell Activation Media. A combination of ZFN mRNA and AAV donor or mRNA donor targeting the same loci (AAVS1, TCRA, CCR5, albumin, HPRT, etc.) was used. The cells were washed 2 times with DPBS then resuspended in BTXpress high performance electroporation solution (Harvard Apparatus, Holliston, Mass.) to a final density of 2.0E+6 cells/mL. Cells (100 μL) and electroporation solution were mixed with ZFN mRNA (4 μg) followed by electroporation in a BTX ECM830 Square Wave electroporator (Harvard Apparatus) in a MOS 96 multi-well Electroporation Plate 2 mm (Harvard Apparatus). Following electroporation cells were transferred to 0.5 mL of B cell Activation Media in a 24 well plate. AAV containing homologous donor templates for target loci was then added at 2.4×10⁶ vg/cell. Plates were gently rocked for 2 minutes. After 2 days, cell culture was harvested, 25 μL of cell culture was collected, mixed with DPBS (175 μL) for flow cytometry analysis, the remaining cell culture was spun down in a table top centrifuge, supernatants collected, and cells washed with DPBS before being transferred to Plasma Blast Generation Media. After 3 days, cell culture was harvested, 25 μL of cell culture was collected, mixed with DPBS (175 μL) for flow cytometry analysis, the remaining cell culture was spun down in a table top centrifuge, supernatants collected and cells washed with DPBS before being transferred to Plasma Cells Generation Media. After 3 days the experiment concluded, cell culture (25 μL from the 500 μL in a single well of a 24-well plate) was collected and mixed with PBS (175 μL) for flow cytometry analysis, the remaining cell culture was spun down in a table top centrifuge, supernatants collected, cells washed with DPBS and harvested for gDNA.

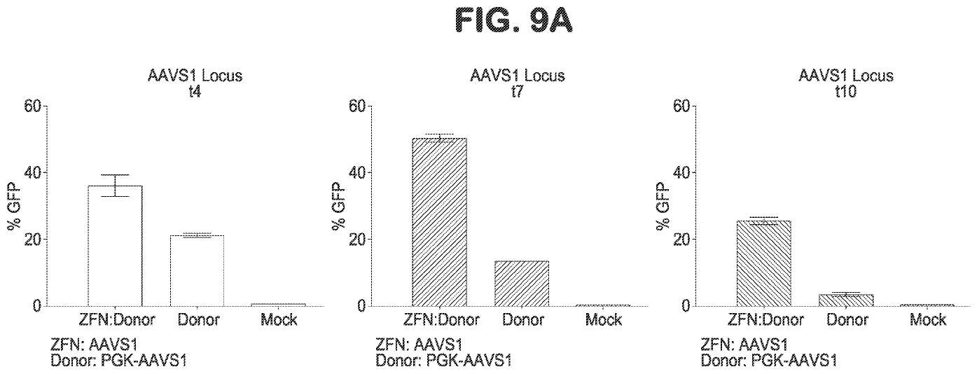

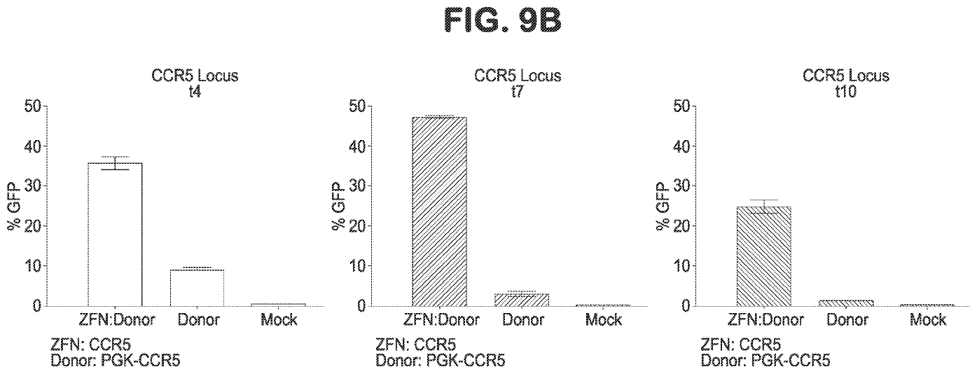

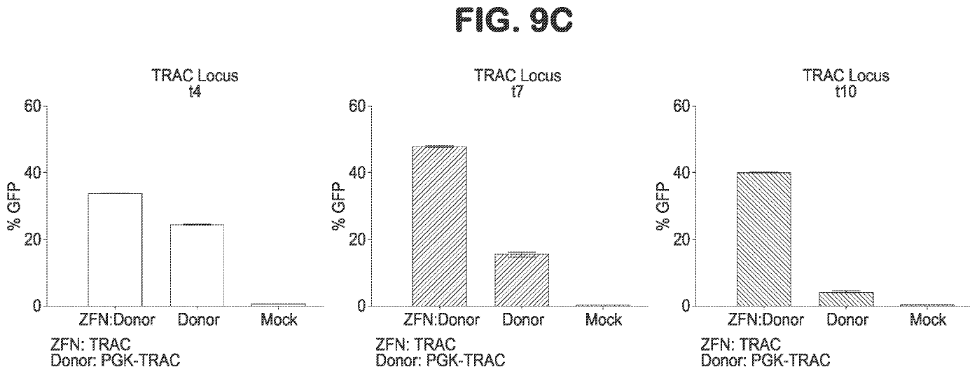

For the flow cytometry, cell culture (25 μL from the 500 μL in a single well of a 24-well plate) was collected and mixed with PBS (175 μL) at days 2, 5 and 8 following the administration of mRNA and AAV donor. GFP expression was analyzed using a Guava EasyCyte 5HT (EMD Millipore, Billerica, Mass., USA). The data was analyzed using InCyte version 2.5 (EMD Millipore). The results (FIGS. 9A through 9C) demonstrate that there was integration of the GFP transgene in all cases, and that use of the specific nucleases lead to the highest percent of GFP positive cells.

To measure target integration of CCR5 and AAVS1 donors, DNA was isolated by a QIAamp DNA mini Kit (Qiagen, Carlsbad Calif.) per the manufacturer's instructions. One hundred nanograms of gDNA was used and a three-step PCR was then carried out using Phusion® Hot Start Flex Polymerase (New England Biolabs, Ipswich, Mass.) and HotStartTaq Master Mix Kit (Qiagen, Carlsbad, Calif.). Illumina deep sequencing measured target integration at each loci. The primers for each step are shown below:

AASV1 Primers:
Step 1 PCR Primers:
AAVS1 Forward 1:
(SEQ ID NO: 16)
5′CGGAACTCTGCCCTCTAACG.
AAVS1 Reverse 1:
(SEQ ID NO: 17)
5′GTGTGTCACCAGATAAGGAATCTG.
Step 2 PCR Primers:
AAVS1 Forward 2:
(SEQ ID NO: 18)
5′CGTCTCTCTCCTGAGTCCG.
AAVS1 Reverse 2:
(SEQ ID NO: 17)
5′GTGTGTCACCAGATAAGGAATCTG.
Step 3 PCR primers:
AAVS1 Forward 3:
(SEQ ID NO: 19)
5′CTCTTTCCCTACACGACGCTCTTCCGATCTNNNNCTCTGGTTCTGG
GTACTTTTATCTG.
AAVS1 Reverse 3:
(SEQ ID NO: 20)
5′AGACGTGTGCTCTTCCGATCTGTGTGTCACCAGATAAGGAATCTG.
AAVS1 wild type amplicon sequence:
(SEQ ID NO: 21)
5′NNNNCTCTGGTTCTGGGTACTTTTATCTGTCCCCTCCACCCCACAG
TGGGGCCACTAGGGACAGGATTGGTGACAGAAAAGCCCCATCCTTAGG
CCTCCTCCTTCCTAGTCTCCTGATATTGGGTCTAACCCCCACCTCCTG
TTAGGCAGATTCCTTATCTGGTGACACAC.
AAVS1 GFP-TI sequence (SEQ ID NO: 22):
5′NNNNCTCTGGTTCTGGGTACTTTTATCTGTCCCCTCCACCCCACAG
TGGGGCAAGCTTCGAGCCATCAGGGCCTGGTTCTTTCCGCCTCAGAAG
GCCTTTTGCAGTTTATCAGGATGAGGATGACCAGCATGTTGCCCACAA
AACCAAAGATGAACACCAGATTCCTTATCTGGTGACACAC
CCR5 Primers
Step 1 PCR Primers:
CCR5 Forward 1:
(SEQ ID NO: 12)
5′GCTCTACTCACTGGTGTTCATCTTT.
CCR5 Reverse 1:
(SEQ ID NO: 7)
5′CTCTGTCTCCTTCTACAGCCAAGC.
Step 2 PCR Primers:
CCR5 Forward 2:
(SEQ ID NO: 12)
5′GCTCTACTCACTGGTGTTCATCTTT.
CCR5 Reverse 2:
(SEQ ID NO: 9)
5′CCAGCAATAGATGATCCAACTCAAATTCC.
Step 3 PCR Primers:
CCR5 Forward 3:
(SEQ ID NO: 23)
5′ACACTCTTTCCCTACACGACGCTCTTCCGATCTNNNNNGCCAGGTT
GAGCAGGTAGATG.
CCR5 Reverse 3:
(SEQ ID NO: 11)
5′AGACGTGTGCTCTTCCGATCTGCTCTACTCACTGGTGTTCATCTT
T.
CCR5 amplicon wild type sequence:
(SEQ ID NO: 12)
5′NNNNNGCCAGGTTGAGCAGGTAGATGTCAGTCATGCTCTTCAGCCT
TTTGCAGTTTATCAGGATGAGGATGACCAGCATGTTGCCCACAAAACC
AAAGATGAACACCAGTGAGTAGAGC
CCR5 TI-GFP sequence:
(SEQ ID NO: 24)
5′NNNNNGCCAGGTTGAGCAGGTAGATGTCAGTCATGCTCTTCAGCCT
TTTGCAGTTTCTCGAGCCATCAGGGCCTGGTTCTTTCCGCCTCAGAAG
TAGAAAGATGAACACCAGTGAGTAGAGC

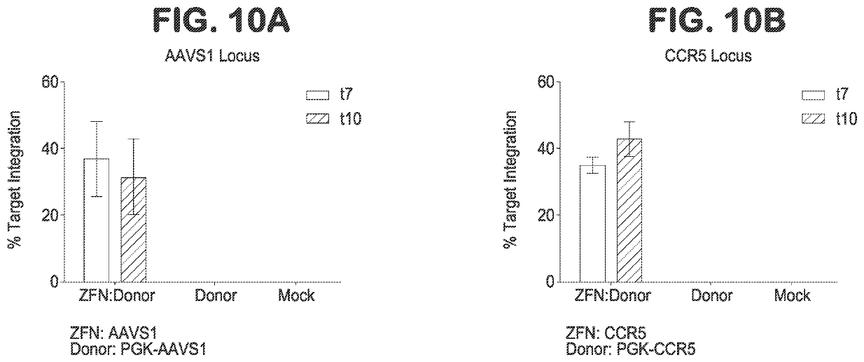

The results (as shown in FIGS. 10A and 10B) demonstrated between 38% and 50% targeted integration for these loci.

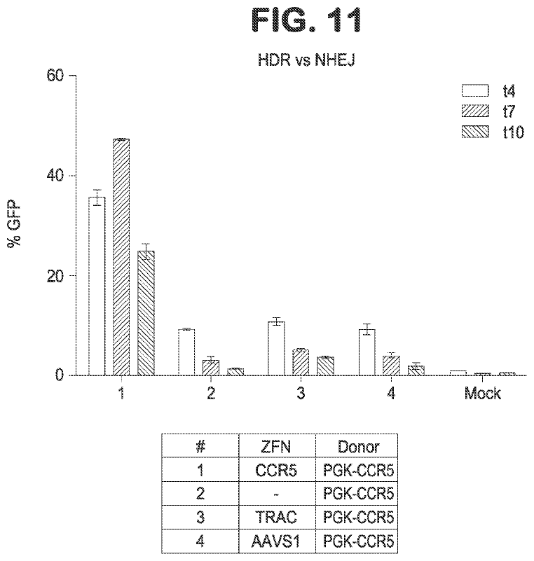

Experiments were also performed using non-matching transgene donors. The homology arms in these non-matching donors did not have homology with sequences flanking the nuclease target site of the co-introduced nuclease. For example, TCRA (TRAC)-specific ZFNs were used in combination with a GFP transgene donor comprising CCR5 homology arms. This donor was also used in combination with AAVS1-specific ZFN. Increased integration found with a matching ZFN target site and donor homology arms would indicate that, at least in part, the transgene integration relies on a homology dependent recombination reaction. The results (FIG. 11) demonstrated that increased donor integration occurs when the donor is flanked by homology arms that match the region surrounding the nuclease cut site, and thus indicate that the cultured B cells predominantly rely on homologous recombination for targeted integration. Integration was seen at a low level for non-matched donors indicating that integration may also occur by end-capture using NHEJ.

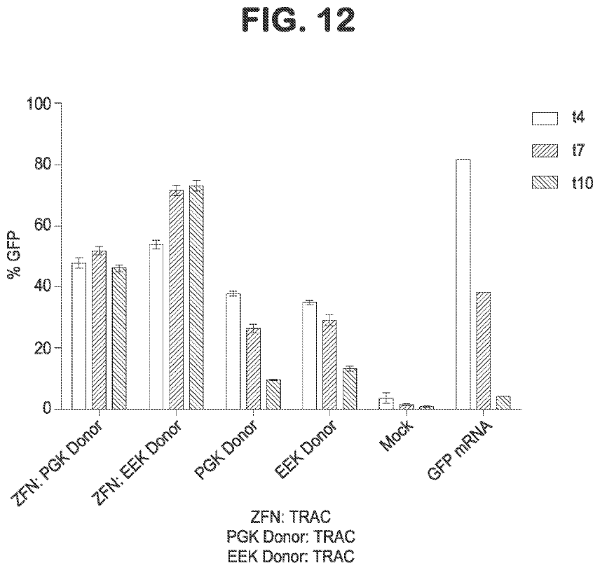

The cells transduced with donor constructs comprising the two alternate promoters were also compared. GFP expression was analyzed by flow cytometry as described above (see FIG. 12) and the results demonstrated that the EEK promoter drove higher GFP expression in the B cells than the PGK promoter.

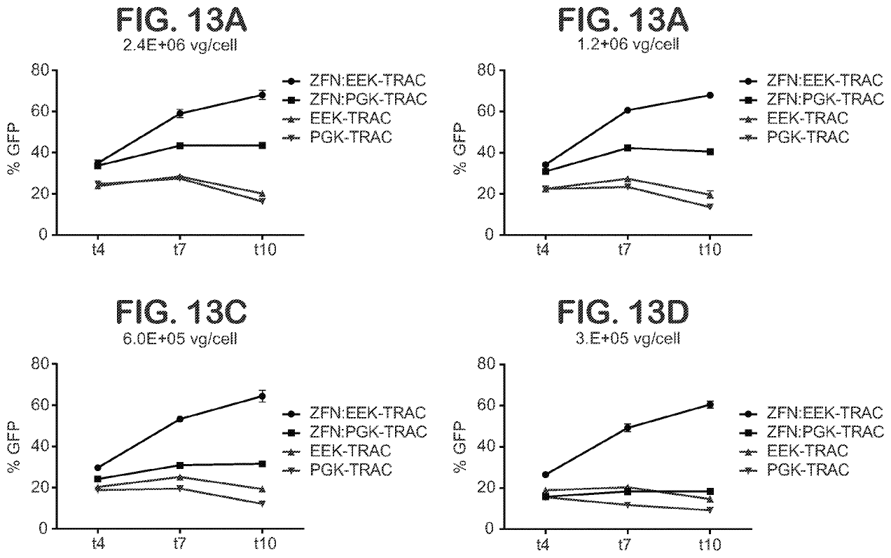

A titration comparing varying amounts of the AAV-donor construct was carried out using a constant dose of ZFN mRNA. The culture B cells were treated with 4μ of TCRA (TRAC)-specific ZFN by electroporation, and then transduced with a range of donor AAV, from 3.0E+05 to 2.4E+06 vg/cell. Furthermore, the two promoters were also compared under these conditions. The results (FIGS. 13A-13D) demonstrated that at the lower donor concentrations, the EEK promoter maintained GFP expression over an 8-fold dilution during the progression of the B cell to plasmablast and plasma cell. The experiment also verified that the use of the nucleases to drive targeted integration resulted in higher GFP expression in B cells.

### Example 7: Antibody Expression During Genome Editing

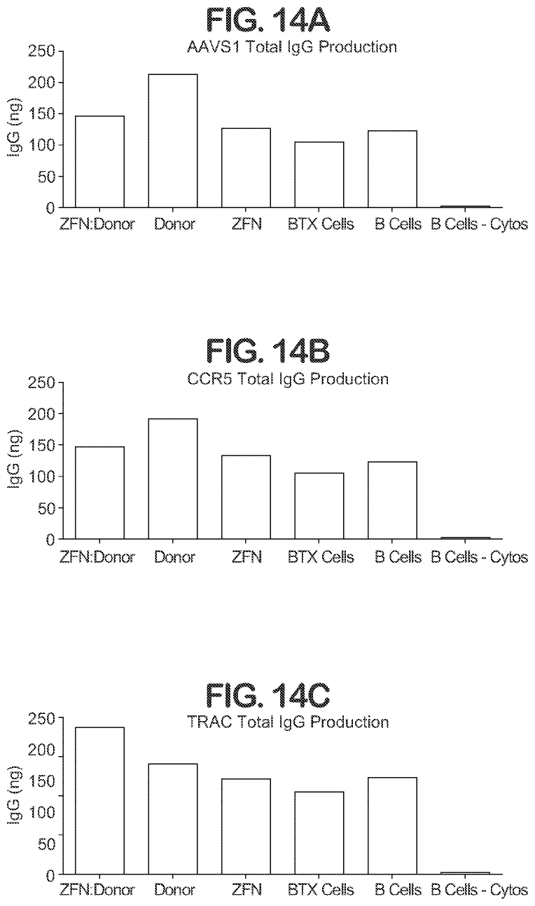

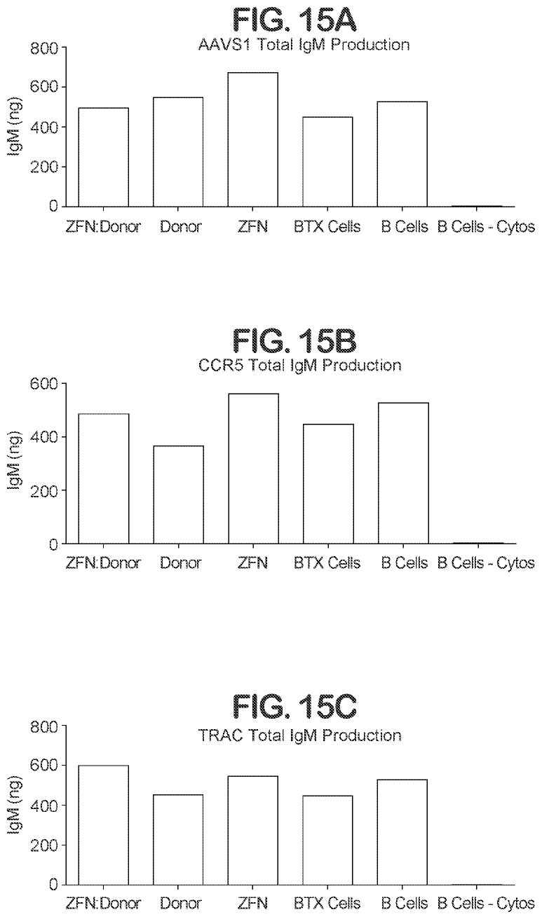

IgG and IgM levels were analyzed by ELISA as described above for the cultured B cells that had undergone electroporation for delivery of the ZFN pairs and also GFP donor. The results (FIGS. 14 and 15) demonstrate that the levels of antibodies produced by the B cells was not highly impacted by electroporation or by electroporation followed by AAV-donor transduction.

### Example 8: Potential Booster Function of AAV in Cultured B Cells

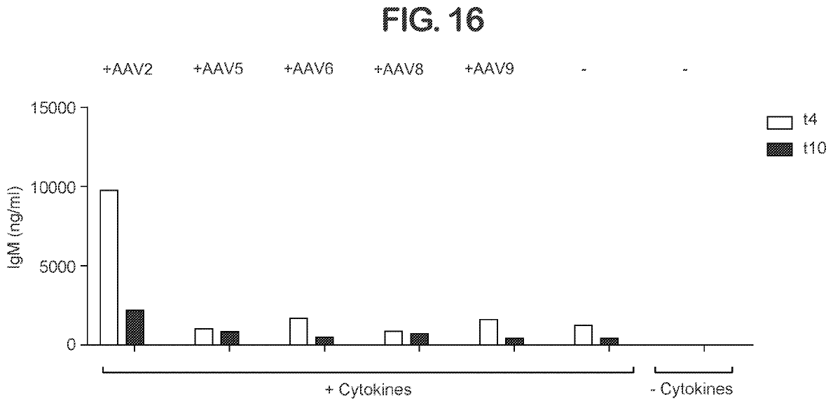

In a cultured B cell that expresses an anti-AAV antibody, it could be possible that re-exposure of that B cell to the AAV serotype that the B cell is reactive against could cause a ‘booster’ effect and induce an increase in anti-AAV antibody production. One CD19+ B cell population from one human donor demonstrated an increase in IgM secretion following treatment with AAV2. Anti-AAV2 antibodies are known to have a robust prevalence in the human population due to the ubiquitous presence of AAV2. Thus, in this study a CD19+ B cell population from one human donor potentially expressing anti-AAV2 antibodies was shown to induce a spike in IgM production following exposure to AAV2, but not other AAV serotypes (FIG. 16).

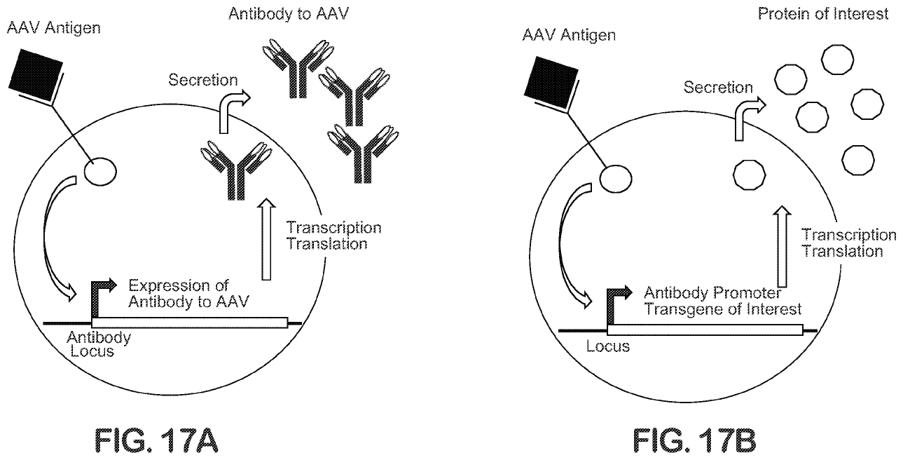

A potential mechanism for the antibody expression spike is shown in FIG. 17, and demonstrates the use of this system for expression of a transgene of interest.

### Example 9: Boost of Transgene Expression Following AAV2 Exposure In Vivo

Transgene donor cassettes are constructed for insertion of a transgene downstream of a B cell promoter. The B cells are treated ex vivo with a specific nuclease, and a donor construct comprising an antibody specific promoter linked to a transgene of interest. B cells chosen for this work are previously verified to produce anti-AAV2 antibodies. The cells are reintroduced into a subject and after a short period of time for engraftment, the subject is treated with AAV2, or AAV2 peptides. The AAV boost upregulates the antibody promoter causing a spike in transgene expression.

### Example 10: B Cell Modification by Targeted Integration of B Cell-Specific Antibody

Transgene donor cassettes (AAV, mRNA, plasmid, etc.) are constructed for insertion of an antibody-encoding transgenes in which the antibody is(are) specific for B cell producing undesirable antibodies (e.g. inhibitors) against a protein delivered by ERT (or an autoantigen), for example B cell producing antibodies against a clotting factor such as F9 (anti-F9 antibodies). Donor cassettes can include homology arms to nuclease target loci (e.g., albumin, TCRA, CCR5, AAVS1, etc.) and are administered in vivo in combination with the suitable nuclease and/or ex vivo to B cell populations (mature, stem and/or B-cell progenitor cell populations) to a subject in need thereof (hemophilia patient with anti-F9 antibodies).

After ex vivo or in vivo modification, the antibody-producing B cells secrete the targeted antibodies which bind to the B cells producing the undesirable antibodies. These targeted antibodies then mediate lysis through mobilization and activation of antibody-dependent cytotoxic cells or though complement mediated lysis. Thus, in the patient these introduced B cells cause a reduction in the endogenous B cells that are producing undesirable antibodies for example, against proteins delivered by ERT or the autoantigen.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

1. A genetically modified B cell comprising one or more modifications comprising:
(a) one or more transgenes, and/or
(b) insertions and/or deletions which modify (i) B cell receptor genes, and/or (ii) cellular interactions in Germinal Centers, and/or
(c) modifications that inhibit suppression of any B cell function associated with pathogen infection or cancer regulation.

2. The genetically modified B cell of claim 1, wherein one or more of the transgenes are integrated into an endogenous locus of the B cell.

3. The genetically modified B cell of claim 1, wherein the transgene encodes a protein lacking or deficient in a subject with a hemophilia, a lysosomal storage disease, a therapeutic antibody and/or a peptide that facilitates crossing the blood-brain barrier when fused to a therapeutic protein.

4. The genetically modified B cell of claim 3, wherein the therapeutic antibody is specific for a B cell that generates inhibitory antibodies to a protein provided by enzyme replacement therapy (ERT) or acts in an autoimmune disease.

5. The genetically modified B cell of claim 3, wherein the therapeutic antibody is specific for a regulatory B cell (Breg) capable of attenuating an anti-tumor response.

6. The genetically modified B cell of claim 4, wherein the protein provided by ERT is a clotting factor.

7. The genetically modified B cell of claim 6, wherein the clotting factor is Factor IX (F9).

8. The genetically modified B cell of claim 1, wherein the transgene further comprises a promoter that drives expression of the transgene.

9. The genetically modified B cell of claim 8, wherein the promoter is a lineage-specific B cell promoter.

10. The genetically modified B cell of claim 1, wherein a transgene is expressed in the cell.

11. The genetically modified B cell of claim 10, wherein the transgene is integrated into a safe harbor locus selected from the group consisting of AAVS1, TCRA, CCR5 or albumin.

12. A genetically modified B cell of claim 1 descended from a genetically modified hematopoietic stem cell.

13. A method of producing a protein in a subject in need thereof, the method comprising administering a population of B cells according to claim 1 to the subject.

14. The method of claim 13, wherein the protein modulates an antibody response in the subject.

15. A method of producing a protein in a subject, the method comprising: introducing into the subject the B cell of claim 1 thereof under conditions such that the B cell produces the protein in the subject.

16. The method of claim 15, wherein the protein is a protein lacking or deficient in a disease or disorder such as a hemophilia or lysosomal storage disease or autoimmune disease or an antibody specific for a B cell producing antibodies to a therapeutic protein supplied in ERT.

17. The method of claim 16, wherein the therapeutic protein supplied in ERT is a clotting factor such as Factor IX (F9) and the antibody is specific for B cells producing anti-clotting factor (anti-F9) antibodies.

18. A kit comprising one or more of the B cells of claim 1.
